(19) United States
(12) Patent Application Publication
(10) Pub. No.: US 2024/0066119 A1
(43) Pub. Date: Feb. 29, 2024

(54) METHODS AND PLATFORMS FOR ELICITING AN IMMUNE RESPONSE IN THE TREATMENT OF CANCER AND COMPOSITIONS AND VACCINES RELATING THERETO

(71) Applicant: University of Maryland, College Park, College Park, MD (US)

(72) Inventors: Christopher M. JEWELL, Silver Spring, MD (US); James I. ANDORKO, Philadelphia, PA (US)

(21) Appl. No.: 18/261,146

(22) PCT Filed: Jan. 12, 2022
(86) PCT No.: PCT/US2022/012080

§ 371 Date: Jul. 12, 2023

Related U.S. Application Data
(60) Provisional application No. 63/265,569, filed on Dec. 16, 2021.
(60) Provisional application No. 63/137,086, filed on Jan. 13, 2021.

(51) Int. Cl. A61K 39/39; A61P 35/00

(52) U.S. Cl. CPC A61K 39/39; A61P 35/00; A61K 2039/545

(57) ABSTRACT

The present disclosure relates to methods for eliciting an immune response to a cancer antigen in a subject in need thereof by introducing directly into at least one lymph node of the subject, and preferably at least two lymph nodes, a therapeutically effective amount of a cancer antigen and/or an adjuvant such that an immune response to the cancer antigen is activated or enhanced in the subject. The present disclosure also concerns pharmaceutical compositions that comprise a therapeutically effective amount of a cancer antigen and/or an adjuvant capable of mediating, and more preferably enhancing, activation of the immune system of a subject against cancer cells that are associated with any of a variety of cancers. The disclosure also relates to the use of such pharmaceutical compositions in the treatment or prevention of a cancer in a recipient subject.

### CROSS-REFERENCE TO RELATED APPLICATIONS

This application is based on U.S. Provisional Patent Application Ser. No. 63/137,086, entitled “System, Materials, and Methods for Geographically Controlling Tumor Growth,” filed Jan. 13, 2021, and U.S. Provisional Patent Application Ser. No. 63/265,569, entitled “Spatial Delivery of Immune Cues as a Platform to Define Therapeutic Outcomes in Cancer Vaccination,” filed Dec. 16, 2021, which applications hereby are incorporated herein by reference in their respective entireties and to which priority is claimed.

### STATEMENT REGARDING FEDERALLY SPONSORED RESEARCH OR DEVELOPMENT

This invention was made with government support under Grant No. CBET1351688 awarded by the National Science Foundation (NSF) and Grant No. R01EB026896 awarded by the National Institutes of Health (NIH). The United States government has certain rights in this invention.

### FIELD OF DISCLOSURE

The present disclosure relates to methods for eliciting an immune response to a cancer antigen in a subject in need thereof by introducing directly into at least one lymph node of the subject, and preferably at least two lymph nodes, a therapeutically effective amount of a cancer antigen and/or an adjuvant such that an immune response to the cancer antigen is activated or enhanced in the subject. The present disclosure also concerns pharmaceutical compositions that comprise a therapeutically effective amount of a cancer antigen and/or an adjuvant capable of mediating, and more preferably enhancing, activation of the immune system of a subject against cancer cells that are associated with any of a variety of cancers. The disclosure also relates to the use of such pharmaceutical compositions in the treatment or prevention of a cancer in a recipient subject.

### BACKGROUND

The body's immune system serves as a defense against a variety of conditions, including, e.g., injury, infection, and neoplasia, and is mediated by two separate but interrelated systems: the cellular and humoral immune systems. Generally, the humoral immune system is mediated by soluble products (antibodies or immunoglobulins) that have the ability to combine with and neutralize structures recognized by the system as being foreign to the body. The cellular immune system involves the mobilization of certain cells (T cells) that serve a variety of therapeutic roles. T cells are lymphocytes that are derived from the thymus and circulate between the tissues, lymphatic system, and circulatory system. They act against, or in response to, a variety of foreign structures (antigens). In many instances, these antigens are expressed on host cells as a result of neoplasia or infection. Although T cells do not themselves secrete antibodies, they are usually required for antibody secretion by the second class of lymphocytes, B cells (which derive from bone marrow). As known in the art, T cells exhibit extraordinary immunological specificity so as to be capable of discerning one antigen from another.

A naive T cell, e.g., a T cell which has not yet encountered its specific antigen, is activated when it first encounters a specific peptide:MHC complex on an antigen-presenting cell (APC). The APC may be a B cell, a macrophage or a dendritic cell. When a naive T cell encounters a specific peptide:MHC complex on an APC, a signal is delivered through the T-cell receptor which induces a change in the conformation of the T cell's lymphocyte function associated antigen (LFA) molecules, and increases their affinity for intercellular adhesion molecules (ICAMs) present on the surface of the APC. The signal generated by the interaction of the T cell with an APC is necessary, but not sufficient, to activate a naive T cell. A second co-stimulatory signal is required. The naive T cell can be activated only by an APC carrying both a specific peptide MHC complex and a co-stimulatory molecule on its surface. Antigen recognition by a naive T cell in the absence of co-stimulation results in the T cell becoming anergic. The need for two signals to activate T cells and B cells such that they achieve an adaptive immune response may provide a mechanism for avoiding responses to self-antigens that may be present on an APC at locations in the system where it can be recognized by a T cell. Where contact of a T cell with an APC results in the generation of only one of two required signals, the T cell does not become activated and an adaptive immune response does not occur.

The efficiency with which humans and other mammals develop an immunological response to pathogens and foreign substances rests on two characteristics: the specificity of the immune response for antigen recognition, and the immunological memory that allows for faster and more vigorous responses upon re-activation with the same antigen (Portolés, P. et al. (2009) “The TCR/CD3 Complex: Opening the Gate to Successful Vaccination,” Current Pharmaceutical Design 15:3290-3300; Guy, C. S. et al. (2009) “Organization of Proximal Signal Initiation at the TCR:CD3 Complex,” Immunol Rev. 232(1):7-21). The specificity of the response of T-cells is mediated by the recognition of antigen (displayed on APCs) by a molecular complex involving the T Cell Receptor (“TCR”) and the cell surface receptor ligand, CD3. The complex is particularly significant since it contains a large number of immunoreceptor tyrosine-based activation motifs (ITAMs).

In mature T cells, TCR/CD3 activation by foreign antigenic peptides associated with self-MHC molecules is the first step needed for the expansion of antigen-specific T cells, and their differentiation into effector or memory T lymphocytes. These processes involve the phosphorylation of the ITAMs of the TCR complex (Guy, C. S. et al. (2009) “Organization of Proximal Signal Initiation at the TCR:CD3 Complex,” Immunol Rev. 232(1):7-21). The outcome of these processes is modulated by the intensity and quality of the antigen stimulus, as well as by the nature of accompanying signals delivered by co-receptor and co-stimulatory surface molecules, or by cytokine receptors (Portoles, P. et al. (2009) “The TCR/CD3 Complex: Opening the Gate to Successful Vaccination,” Current Pharmaceutical Design 15:3290-3300; Riha, P. et al. (2010) “CD28 Co-Signaling In The Adaptive Immune Response,” Self/Nonself 1(3): 231-240).

The growth and metastasis of tumors depend to a large extent on their capacity to evade host immune surveillance and overcome host defenses. Most tumors express antigens that can be recognized to a variable extent by the host immune system, but in many cases, an inadequate immune response is elicited because of the ineffective activation of effector T cells (Khawli, L. A. et al. (2008) “Cytokine, Chemokine, and Co-Stimulatory Fusion Proteins for the Immunotherapy of Solid Tumors,” Exper. Pharmacol. 181:291-328). Thus, although the adaptive immune system can be a potent defense mechanism against disease, it is often hampered by immune suppressive mechanisms in the tumor microenvironment, such as the expression of co-inhibitory molecules. Furthermore, co-inhibitory molecules expressed by tumor cells, immune cells, and stromal cells in the tumor milieu can dominantly attenuate T-cell response against cancer cells.

Vaccines combat disease through molecularly-specific mechanisms that enable both selective and long-lasting outcomes. These technologies have helped catalyze breakthrough cancer immunotherapies, such as chimeric antigen receptor (CAR) T cells that are providing remarkable impacts for certain patient groups (Porter, D. L. et al. (2011) Chimeric Antigen Receptor-Modified T Cells in Chronic Lymphoid Leukemia. New Eng J Med 365:725-733; Maude, S. L. et al. (2014) Chimeric Antigen Receptor T Cells for Sustained Remissions in Leukemia. New Eng J Med 371:1507-1517; Klichinsky, M. et al. (2020) Human chimeric antigen receptor macrophages for cancer immunotherapy. Nat Biotechnol, doi:10.1038/s41587-020-0462-y). CAR T cell therapy involves isolation, engineering, expansion, and reinfusion of a patient's own immune cells. Another technology in cancer research is cancer vaccination. The first clinical example, PROVENGE®, involves complex engineering of a patient's own APCs to generate an important, but modest therapeutic impact (Jou, K. J. et al. (2021) The Changing Landscape of Therapeutic Cancer Vaccines-Novel Platforms and Neoantigen Identification. Clin Cancer Res 27:689-703). Unfortunately, these cell-therapy techniques are complex, requiring sophisticated and expensive manufacturing and regulatory regimens, e.g., with costs for isolating and engineering patient's cells ex vivo as high as $500,000 per treatment round (Singh, A. K. & McGuirk, J. P. (2020) CAR T cells: continuation in a revolution of immunotherapy. Lancet Oncol 21:e168-e178). Thus, simpler and less expensive approaches that provide a similar vaccine-like specificity (and without complex and expensive isolation, engineering, and reinfusion of patient cells) are needed.

As with traditional vaccines, cancer vaccines are comprised of fragments of tumors or cancerous cells (cancer antigens) mixed with stimulatory adjuvants in an attempt to drive strong, lasting responses against the tumor antigen. Adjuvants take a range of forms that mimic molecular patterns of pathogens such as toll-like receptor (TLR) agonists, insoluble particulates of aluminum salts, or physical moieties foreign to the host (O'Hagan, D. T. & Fox, C. B. (2015) New generation adjuvants—From empiricism to rational design. Vaccine 33:B14-B20; Moyer, T. J. et al. (2016) Beyond antigens and adjuvants: formulating future vaccines. J Clin Invest 126:799-808; Cheung, A. S. & Mooney, D. J. (2015) Engineered materials for cancer immunotherapy. Nano Today 10:511-531; Davitt, C. J. H. & Lavelle, E. C. (2015) Delivery strategies to enhance oral vaccination against enteric infections. Adv Drug Deliv Rev 91:52-69). However, anatomical geography also plays a crucial role in determining effectiveness (Zinkernagel, R. M. et al. (1997) Antigen localisation regulates immune responses in a dose-and time dependent fashion: a geographical view of immune reactivity. Immunological Reviews 156:199-209; Ochsenbein, A. F. et al. (2001) Roles of tumour localization, second signals and cross priming in cytotoxic T-cell induction. Nature 411:1058-1064), as vaccine and immunotherapy components must reach specialized immune tissues such as lymph nodes (LNs).

LNs integrate combinations and concentrations of immune signals to coordinate appropriate antigen-specific responses. In LNs, APCs present antigen—both antigen that passively drains to LNs and antigen that is carried to LNs by APCs—to the resident T and B lymphocytes responsible for the specific nature of the response. For example, while traditional preventative vaccines seek long-lasting memory function, therapeutic vaccines would benefit from initially inducing rapid and intense effector function. More sophisticated insight into how immune signals are integrated in LNs to drive antigen specific responses tailored for immunotherapy would therefore enable next generation therapeutic vaccines for cancer (see Blass, P. A. et al. (2021) Advances in the development of personalized neoantigen-based therapeutic cancer vaccines. Nat Rev Clin Oncol 18:215-229; Hu, Z. et al. (2018) Towards personalized, tumour-specific, therapeutic vaccines for cancer. Nat Rev Immunol 18:168-182; Zhang, C. L. et al. (2019) Advances in mRNA Vaccines for Infectious Diseases. Front. Immunol. 10:13).

Biomaterials offer a unique opportunity to engineer a delivery system with programmable parameters such as size (e.g., nano- or micro-scale), surface properties (e.g., charge, topography), and the release of included cargo over time (e.g., degradation, enzymatically-triggered) (Andorko, J. I. & Jewell, C. M. (2017) Designing biomaterials with immunomodulatory properties for tissue engineering and regenerative medicine. Bioeng Transl Med 16(2):139-155). This distinctive control that biomaterials provide has motivated pre-clinical attempts to use carriers and scaffolds to direct cancer immunotherapy (Gosselin, E. A. et al. (2018) Designing natural and synthetic immune tissues. Nature Materials 17:484-498; Eppler, H. B. & Jewell, C. M. (2020) Biomaterials as Tools to Decode Immunity. Adv Mater 32:26; Najibi, A. J. & Mooney, D. J. (2020) Cell and tissue engineering in lymph nodes for cancer immunotherapy. Adv Drug Deliv Rev 161:42-62; M. P. Manspeaker & S. N. Thomas (2020) Lymphatic immunomodulation using engineered drug delivery systems for cancer immunotherapy. Adv Drug Deliv Rev 160:19-35; N. Gong, N.C. et al. (2021) Nanomaterials for T-cell cancer immunotherapy. Nat Nanotechnol 16:25-36), and recently, in clinical trials (F. S. Hodi et al., Dendritic Cell Activating Scaffold in Melanoma, ClinicalTrials.gov, NCT01753089, 2017).

The central role of LNs to generate tumor-specific immune responses has motivated much of the prior work. Conventional strategies provide for systemic injection of nanoparticles with chemical modifications introduced to engage natural host trafficking mechanisms to shuttle immune cues to LNs (Liu, H. et al. (2014) Structure-based programming of lymph-node targeting in molecular vaccines. Nature 507:519-522; see also Reddy, S. T. et al. (2006) In vivo targeting of dendritic cells in lymph nodes with polypropylene sulfide) nanoparticles. J Control Release 112:26-34; Reddy, S. T. et al. (2006) Targeting dendritic cells with biomaterials: developing the next generation of vaccines. Trends in Immunology 27:573-579; Reddy, S. T. et al. (2007) Exploiting lymphatic transport and complement activation in nanoparticle vaccines. Nat Biotechnol 25:1159-1164; Cubas, R. et al. (2009) Virus-like particle (VLP) lymphatic trafficking and immune response generation after immunization by different routes. J Immunol 32:118-128), or that can be carried to LNs by APCs that engulf vaccines (Kwon, Y. J. et al. (2005) In vivo targeting of dendritic cells for activation of cellular immunity using vaccine carriers based on pH-responsive microparticles. PNAS 102:18264-18268; van Broekhoven, C. L. et al. (2004) Targeting dendritic cells with antigen-containing liposomes: a highly effective procedure for induction of antitumor immunity and for tumor immunotherapy. Cancer Res 64:4357-4365).

However, methodologies providing for systemic injections of immunotherapies are limited by the efficiency of targeting to LNs or tumors, and equally importantly, the toxicity or off-target effects of potent immune drugs. The injected dose of these therapies must reach LNs and be retained in these tissues at immunologically-relevant concentrations and time scales without distribution or drainage to other tissues or organs to avoid adverse off-target effects and toxicity (D. J. Irvine & E. L. Dane (2020) Enhancing cancer immunotherapy with nanomedicine. Nat Rev Immunol 20(5):321-334; M. J. O'Melia et al. (2020) Quality of CD8⁺ T cell immunity evoked in lymph nodes is compartmentalized by route of antigen transport and functional in tumor context. Sci Adv 6(50): eabd7134).

Unfortunately, such conventional treatment schemes involving systemic injection have faced substantial challenges and disadvantages. Alternative methodologies seek to exploit the geographic interplay between LNs, tumors, and the location of antigens, adjuvant, or other signals (R. S. Riley et al. (2019) Delivery technologies for cancer immunotherapy. Nat Rev Drug Discov 18(3):175-196; X. Duan et al. (2019) Nanoparticle-Mediated Immunogenic Cell Death Enables and Potentiates Cancer immunotherapy. Angew Chem Int Ed Engl 58(3):670-680; M. S. Goldberg (2019) Improving cancer immunotherapy through nanotechnology. Nat Rev Cancer 19(10):587-602). For example, compared with systemic injection, direct intratumoral injection and intradermal injection of immune checkpoint inhibitors have exhibited some improvement in outcomes in pre-clinical models (D. M. Francis et al. (2020) Blockade of immune checkpoints in lymph nodes through locoregional delivery augments cancer immunotherapy. Sci Transl Med 12(563):eaay3575).

Likewise, nanoparticle-based strategies have been improved with similar loco-regional tumor injection approaches (J. Kim et al. (2020) Poly(cyclodextrin)-Polydrug Nanocomplexes as Synthetic Oncolytic Virus for Locoregional Melanoma Chemaimmunotherapy. Adv Funct Mater 30(16):1908788; M. A. Aznar et al. (2019) Immunotherapeutic effects of intratumoral nanoplexed poly I:C. J Immunother Cancer 7(1):116). In these processes, the route of antigen transport to and within LNs are important in determining outcomes (M. J. O'Melia et al. (2020) Quality of CD8⁺ T cell immunity evoked in lymph nodes is compartmentalized by route of antigen transport and functional in tumor context. Sci Adv 6(50):eabd7134). Another spatially-localized cancer immunotherapy is now testing a biomaterial scaffold for melanoma in clinical trials, wherein tumor cells are isolated and lysed, then scaffolds are loaded with lysate, adjuvants, and immune cell-recruiting molecules prior to implantation within the tumor. The implanted scaffold then recruits APCs to sample patient-derived antigen within the scaffold, and ideally migrates to LNs to generate tumor-specific adaptive responses. Together, these examples highlight the distinct potential of “geographic” immunotherapy in the context of solid tumors or other diseases with geographic localization.

An intriguing delivery method for geographic immunotherapy involves the use of ultrasound guidance or lymphatic tracers. Delivery of soluble antigen to LNs has been tested in immunotherapy contexts (D. Weinfeld et al. (2020) A preseason booster prolongs the increase of allergen specific IgG4 levels, after basic allergen intralymphatic immunotherapy, against grass pollen seasonal allergy. Allergy, Asthma Clin Immunol 16:31; Ribas, A. et al. (2011) Intra-lymph node prime-boost vaccination against Melan A and tyrosinase for the treatment of metastatic melanoma: results of a phase 1 clinical trial. Clin Cancer Res 17:2987-2996; Adamina, M. et al. (2010) Intranodal immunization with a vaccinia virus encoding multiple antigenic epitopes and costimulatory molecules in metastatic melanoma. Mol Ther 18:651-659; G. Senti et al. (2008) Intralymphatic allergen administration renders specific immunotherapy faster and safer: a randomized controlled trial. PNAS 105 (46): 17908-17912; G. Senti et al. (2012) Intralymphatic immunotherapy for cat allergy induces tolerance after only 3 injections. J Allergy Clin Immunol 129(5):1290-1296; Y. Waeckerle-Men et al. (2013) Lymph node targeting of BCG vaccines amplifies CD4 and CD8 T-cell responses and protection against Mycobacterium tuberculosis. Vaccine 31:1057-1064; Johansen, P. et al. (2005) Direct intralymphatic injection of peptide vaccines enhances immunogenicity. Eur J Immunol 35:568-574). By combining a geographic immunotherapy approach with polymer depots, this antigen delivery strategy has shown some promise to drive immune tolerance (J. I. Andorko et al. (2015) Harnessing biomaterials to engineer the lymph node microenvironment for immunity or tolerance. AAPS J 17(2):323-328; Gammon, J. M. et al. (2015) Controlled delivery of a metabolic modulator promotes regulatory T cells and restrains autoimmunity. J Control Release 210:169-178; Tostanoski, L. H. et al. (2016) Reprogramming the Local Lymph Node Microenvironment Promotes Tolerance that Is Systemic and Antigen Specific. Cell Rep 16:2940-2952).

Despite the array of approaches, biomaterials have been underutilized as tools to determine and utilize immune signaling and communication in and between LNs (Eppler, H. B. & Jewell, C. M. (2020) Biomaterials as Tools to Decode Immunity. Adv Mater 32:26). In addition, a need remains for improved therapies utilizing biomaterials for treating cancers, e.g., treatment regimens that facilitate a targeted immune response against cancers by mediating T-cell activation, enhancing an immune response and/or killing cancer cells.

### SUMMARY OF THE DISCLOSURE

The present disclosure relates to methods, materials, and compositions for geographically controlling and/or preventing tumor growth. In some embodiments, the disclosed methods, materials, and compositions include the introduction of polymer scaffolds directly into lymph nodes (LNs) via intra-lymph node injection. In some embodiments, the polymer scaffolds comprise stimulatory immune cues and/or peptide antigens, e.g., a cancer antigen(s). In some embodiments, the disclosed methods, materials, and compositions are used to locate where in a subject signals and tumors reside. In some embodiments, the disclosed systems, materials, and methods are used to retard or prevent the growth of tumors in a desired area.

Intra-LN injection of biomaterial carriers or depots provides a unique tool to directly study pro-immune signal integration during cancer immunotherapy: the ability to study responses and communication between injected LNs, non-injected LNs, and how these responses may vary when proximal or distal to tumors. This level of precision is difficult to achieve using peripheral injections, which rely on passive or active drainage of carriers and signals to LNs and again are limited by the percentage of total dose delivered to the LN. The methods and systems of the present disclosure provide for the direct introduction of size-restricted degradable depots that are too large to drain from LNs after intra-LN injection, thereby bypassing the need for lymphatic draining and APC targeting. Potent therapeutic treatment strategies are achieved, which generate tumor-specific immune cells without systemically exposing patients to potentially toxic immunotherapeutics.

The present disclosure relates to a method of eliciting an immune response to a cancer antigen in a subject comprising introducing directly into at least one lymph node of the subject: a therapeutically effective amount of a composition comprising a cancer antigen, in combination with a carrier comprising an adjuvant such that an immune response to said cancer antigen is activated or enhanced in the subject.

The present disclosure also relates to a method of treating or preventing cancer in a subject in need thereof, comprising introducing directly into at least one lymph node of the subject: a therapeutically effective amount of a composition comprising a cancer antigen, in combination with a carrier comprising an adjuvant such that an immune response to said cancer antigen is activated or enhanced in the subject.

The present disclosure also relates to pharmaceutical compositions comprising a therapeutically effective amount of a cancer antigen, an adjuvant formulated to activate or enhance an immune response to the cancer antigen in a subject, and a pharmaceutically acceptable carrier, diluent, and/or excipient. Preferably, the pharmaceutical composition is formulated for administration via intra-lymph injection.

In some embodiments, the carrier is a microparticle. In some embodiments, the microparticle has a diameter of between about 1 μm and about 10 μm. In some embodiments, the microparticle is biodegradable and/or biocompatible. The microparticle may comprises a polymer material, e.g., including but not limited to poly(glycolide) (PGA), poly(L-lactide) (PLA), poly(beta-amino esters), and polyethylene glycol (PEG). In some embodiments, the adjuvant is a toll-like receptor (TLR) agonist. In some embodiments, the TLR agonist is polyinosinic: polycytidylic acid (Poly(I:C)).

In some embodiments, the cancer antigen and/or the adjuvant is introduced directly into the at least one lymph node of the subject via intra-lymph node injection. In some embodiments, the cancer antigen and the adjuvant are introduced into a single lymph node of the subject. In some embodiments, the cancer antigen and the adjuvant are introduced into at least two lymph nodes of the subject. In some embodiments, the cancer antigen is introduced into one or more lymph node(s) of the subject, and the adjuvant is introduced into one or more lymph node(s) of the subject different from the one or more lymph node(s) into which the cancer antigen is introduced. In some embodiments, the lymph node(s) into which the adjuvant is introduced is a/are tumor draining lymph node(s).

In some embodiments, the cancer antigen is associated with a cancer selected from the group consisting of breast cancer, prostate cancer, lung cancer, stomach cancer, colon cancer, rectal cancer, pancreatic cancer, liver cancer, ovarian cancer, throat cancer, esophageal cancer, bone cancer, melanoma, uterine cancer, testicular cancer, bladder cancer, kidney cancer, brain cancer, thyroid cancer, lymphoma, and leukemia. In particular embodiments, the cancer is a melanoma or a lymphoma, and/or the subject is a human.

### BRIEF DESCRIPTION OF THE DRAWINGS

FIG. 1. PolyIC MP characterization. Representative data showing particle size (a), zeta potential (b), PolyIC loading (normalized to Empty MP) (c), and PolyIC release in media over time (d) following particle synthesis. SEM micrographs of Empty (e) and PolyIC MPs (f) are shown. Data represents three separate particle batches (panels a-d) and errors bars represent standard deviation. SEM images (panels e-f) display scale bars of 20 μm (left) and 10 μm (right), respectively.

FIG. 2. Immune cargos can be localized to different LNs, resulting in varied initial immune responses. (a) Schematic representation of treatment scheme where vaccine components are localized in overall matched doses given entirely to one LN (“One LN,” left), given in half doses to two inguinal LNs (“Two LN,” center), or split with the adjuvant-containing MPs in one LN and the antigen in the other (“Split LN,” right). (b) Representative fluorescent micrographs showing the colocalization of immune signals to LNs 24 hours after treatment via One LN injection. (PolyIC MP—red stain areas identified by numeral ‘1’; FITC-OVA—green stain areas shown in gray). Scale bar=200 μm. (c) Representative IVIS images (left) of “One LN” and “Split LN” treated mice and (d) aggregate data for PolyIC MP deposition in LNs. Dashed gray line indicates the average total radiant efficiency for the “Sham” treatment group. N=3 mice per group and errors bars represent SEM. (Using a One-way ANOVA with a Tukey post-test, * p<0.05 when comparing left (L) to right (R) LN within the same treatment.) (e) Representative contour plots and (f) quantification of SIINFEKL-specific CD8⁺ T cells in blood 7 days after treatment as in (a). n=4 per group for Day 14, 21, and 28 and errors bars represent SEM. (Using a One-way ANOVA with a Tukey post-test, * p<0.05; ** p<0.01; *** p<0.001, ****p<0.0001 compared to Sham treatment, p<0.0001 compared to Empty). (g) Evaluation of SIINFEKL-specific CD8⁺ T cells in the blood over time. N=8 mice per group for Day 0 and 7.

FIG. 3. Sham treated LN images. Representative fluorescent micrographs of a Sham treated LN showing no colocalization of immune signals to LNs 24 hours after treatment. (PolyIC MP; FITC-OVA). Scale bar=200 μm.

FIG. 4. Rapid uptake of immune signals by innate immune cells within LNs leads to local activation. The overall percentage of cells (a) and number of cells (b) that were able to take up particles following “One LN,” “Two LN,” or “Split LN” treatment 24 hours after intra-LN treatment. (c) Representative gating scheme used to determine innate cell subpopulations including macrophages (CD11b⁺/CD11c⁻, F4/80⁺/Ly6G⁻) and dendritic cells (DCs) (CD11c⁺) which were further characterized as plasmacytoid DCs (CD11c⁺, B220⁺), CD8⁺ DCs (CD11c⁺, B220⁻/CD8⁺) or the remaining LN resident/migratory conventional DCs (CD11c⁺, B220⁻/CD8⁻). (d) Percentage of cells containing particles within DC parent (left) and subpopulations (right of dotted line). (e) Number of activated DCs (CD11c⁺) 7 days after treatment. For all panels, n=4 mice per group and errors bars represent SEM. (Using a One-way ANOVA with a Tukey post-test, * p<0.05; ** p<0.01; *** p<0.001, ****p<0.0001 compared to Sham treatment).

FIG. 5. Particle uptake and macrophage activation. MFI (a) and percentage (b) of cells containing particles for each treatment, irrespective of cell subpopulations. (c) Representative gating scheme for innate cell activation; top shows identification of DCs and macrophages, bottom shows gating of activation markers. (d) Number of activated macrophages (F4/80+) 7 days after treatment. N=4 mice per group and errors bars represent SEM. (Using a One-way ANOVA with a Tukey post-test, * p<0.05; ** p<0.01; *** p<0.001, ****p<0.0001 compared to Sham treatment).

FIG. 6. Vaccine location dictates the antigen-specific responses within the LN. (a) Schematic representation of vaccine treatment with experimental readouts and (b) representative gating scheme to determine CD8⁺, tetramer-positive (Tet⁺) cells in LNs. Evaluation of SIINFEKL-specific CD8⁺ T cells within the treated LNs of mice as treated (a) at Day 7 (left) and Day 28 (right) post-treatment by percentage (c) and MFI (d) within the CD8⁺/Tet⁺ gate. For all panels, n=4 mice per group and errors bars represent SEM. (Using a One-way ANOVA with a Tukey post-test, * p<0.05; ** p<0.01; *** p<0.001, ****p<0.0001 compared to Sham treatment).

FIG. 7. Split LN treatment relies on adjuvant treatment in LN to enhance antigen specificity and is dependent on cellular trafficking. (a) Schematic representation of treatment schedule and experimental readouts for studies comparing “Split LN” with OVA contralateral to phosphate-buffered saline (PBS) or PolyIC MPs. (b) Evaluation of SIINFEKL-specific CD8⁺ T cells in the blood following treatment in (a). Percentage (c) and number (d) of SIINFEKL-specific CD8⁺ T cells in the treated LNs following treatment as in (a). (e) Schematic representation of treatment schedule and experimental readouts for studies with FTY720 treatment to inhibit lymphocyte egress from lymphoid tissues. Percentage (f) and number (g) of SIINFEKL-specific CD8⁺ T cells in blood following treatment in (e). (h) Number of SIINFEKL-specific CD8⁺ T cells in LNs following treatment in (e). (i) Schematic representation of treatment schedule and experimental readouts for studies evaluating chemokine expression. Percentage of CCR7⁺ (j) and CCR5⁺ (k) among antigen-specific, CD8⁺ cells following intra-LN treatment; Sham treatment groups were omitted from this analysis among antigen-specific T cells since these animals did not receive a vaccine and did not exhibit measurable antigen-specific CD8⁺ cells. (panels: b-d) n=6 mice per group and errors bars represent SEM. (Using a One-way ANOVA with a Tukey post-test, * p<0.05; ** p<0.01; *** p<0.001, ****p<0.0001 compared to Sham treatment). (panels: f-h) n=4 mice per group and errors bars represent SEM. (Using a Two-way ANOVA with a Tukey post-test, * p<0.05; ** p<0.01; *** p<0.001, ****p<0.0001 compared to matched treatment without FTY720). (panels: j-k) n=4 mice per group and errors bars represent SEM. (Using a One-way ANOVA with a Tukey post-test, * p<0.05; ** p<0.01; *** p<0.001, ****p<0.0001 as indicated by brackets).

FIG. 8. Chemokine gating scheme and quantification. Representative gating scheme for chemokine staining in treated LNs with CD8, Tet, CCR5, and CCR7.

FIG. 9. Geography of vaccination dictates survival following initial inoculation and rechallenge in a B16-OVA melanoma model. (a) Schematic representation of “Two LN,” “One LN,” or “Split LN” treatment and subsequent tumor inoculation in proximity to tumor-draining LNs (tdLN) and non-tumor-draining LNs (non-tdLN). (b) Schematic representation of treatment schedule and experimental readouts. (c) Traces of tumor volumes for individual mice with number of mice surviving indicated. (d) Incidence and (e) survival of mice following treatment in (b). (f) Percentage of mice which survived the initial inoculation that failed to grow a secondary tumor following a re-challenge on Day 91 (98 days after initial vaccination) with 100,000 B16-OVA cells. (panels a-e) n=6 mice per group. (panel d-e) Statistics indicated in Table 1. Mice per group indicated in Table 2.

FIG. 10. Presence of OVA and PolyIC MP within the same treated LNs decreases tumor burden, incidence, and increases survival in a lymphoma model. (a) Schematic representation of treatment schedule and experimental readouts. (b) Traces of tumor volumes for individual mice with number of mice surviving indicated. (c) Incidence and (d) survival of mice following treatment as in (a). (panels c-f) n=6 mice per group and errors bars represent SEM. (panel c-d) Statistics indicated in Table 3.

FIG. 11. Antigen specific T cell responses in blood during tumor models. (a) Evaluation of SIINFEKL-specific CD8⁺ T cells in the blood at day 7 and (b) over time following treatment as in FIG. 9b with melanoma model. (c) Evaluation of SIINFEKL-specific CD8⁺ T cells in the blood at day 7 and (d) over time following treatment as in FIG. 10a with lymphoma model. (panels a-d) N=6 mice per group and errors bars represent SEM. (Using a One-way ANOVA with a Tukey post-test, * p<0.05; ** p<0.01; *** p<0.001, ****p<0.0001 compared to Sham treatment unless otherwise noted).

FIG. 12. Evaluating intranodal injection as a therapeutic treatment in a melanoma model. (a) Schematic representation of “subcutaneous (s.c.) (tdLN)” or “One LN (tdLN)” treatment in proximity to tumor-draining LNs (tdLN) and (b) treatment schedule. (c) Tumor burden at day 15 post tumor inoculation where no statistical significance was seen between groups. (d) Survival of mice following treatment in (a) (panels c-d) n=8 mice per group and errors bars represent SEM. (panel c: using a Student's t tests and panel d: using survival curve analysis, * p<0.05 compared to s.c. treatment).

FIG. 13. Schematic showing intra-LN injection strategies into model including deposition of dose-matched treatments to a single LN, split equally in two LNs, or segregated with antigen and adjuvant in separate LNs.

### DETAILED DESCRIPTION OF EMBODIMENTS

The present disclosure encompasses therapies for treating a cancer and/or for activating or enhancing an immune response against a cancer antigen. As used herein, the terms “treatment” or “treating” refer to an approach for obtaining a beneficial or desired result, and preferably a beneficial or desired clinical result. Such beneficial or desired clinical result includes, but is not limited to, one or more of the following: shrinking the size of a tumor, retardation of cancer cell growth, delaying the development of metastasis, decreasing a symptom resulting from the disease, increasing the quality of life of those suffering from the disease, decreasing the dose of other medications required to treat the disease, enhancing the effect of another medication such as via targeting and/or internalization, delaying the progression of the disease, and/or prolonging survival of an individual. Subjects for treatment include animals, most preferably mammalian species such as non-primate (e.g., bovine, equine, feline, canine, rodent, etc.) or a primate (e.g., monkey, such as, cynomolgus monkey, human, etc.). In a preferred embodiment, the subject is a human.

In accordance with disclosed embodiments, a therapeutically effective amount of a cancer antigen and an adjuvant is administered to a subject in need thereof, such that an immune response to the cancer antigen is activated or enhanced in the subject. The cancer antigen and adjuvant are preferably introduced directly into at least one lymph node(s) of the subject. The present disclosure also relates to pharmaceutical compositions that comprise a therapeutically effective amount of a cancer antigen and an adjuvant capable of mediating, and more preferably enhancing, the activation of the immune system against the cancer antigen that is associated with any of a variety of human cancers. The disclosure also relates to the use of such pharmaceutical compositions to treat and/or prevent cancer and other diseases in a recipient subject.

Exemplary diseases and conditions that may be treated by the methods and compositions of the present disclosure include, but are not limited to, proliferative disorders, cell proliferative disorders, and cancer (especially a melanoma or a lymphoma). Thus, the disclosure encompasses methods and compositions for the treatment, prevention or management of a cancer, comprising administering to the subject via direct injection into at least one lymph node(s) of the subject a therapeutically effective amount of a cancer antigen and an adjuvant such that an immune response to the cancer antigen is activated or enhanced in the subject. Although not intending to be bound by a particular mechanism of action, the introduction of such molecules directly into the lymph node(s) of a subject mediates effector function against cancer cells, promotes the activation of the immune system against cancer cells, and enhances apoptosis or negative growth regulatory signaling, or a combination thereof, resulting in tumor clearance and/or tumor reduction.

The methods and compositions of the present disclosure may be used for immunotherapy directed at various cancers, e.g., including but not limited to: breast cancer, prostate cancer, gastric cancer, lung cancer, stomach cancer, colon cancer, rectal cancer, pancreatic cancer, liver cancer, ovarian cancer, pharyngeal cancer, esophageal cancer, laryngeal cancer, bone cancer, skin cancer, melanoma, uterine cancer, testicular cancer, bladder cancer, kidney cancer, brain cancer, thyroid cancer, lymphoma, and leukemia. The methods and compositions of the present disclosure may, for example, delay the development of metastasis and/or to promote the activity of the immune system against the cancer cells.

The methods and compositions of the present disclosure may utilize or include a cancer antigen associated with a particular cancer. Numerous antigens associated with cancers are known in the art and include cell surface tumor antigens or epitopes (such as: 17-1A, A33, adult erythrocyte primary endoderm I antigen, alpha fetoprotein, an envelope antigen of an RNA tumor virus, bladder tumor oncofetal antigen, B7-H1, B7-H2, B7-H3, B7-H4, B7-H5, B7-H6, Burkitt's lymphoma antigen-38.13, CA125, CD18, CD19, human B-lymphoma antigen-CD20, CD22, CD33, CD44, CD52, CEA, CO17-1A, CTA-1, CTLA-4, epidermal growth factor receptor, Ep-CAM, EphA2, fetal erythrocyte I antigen, fibrosarcoma antigen, ganglioside GD2, ganglioside GD3, ganglioside GM2, ganglioside GM3, GICA 19-9, gp IIIb/IIIa, gp72, HER1, HER-2/neu, HER3, HER4, high molecular weight melanoma antigen, HLA-DR antigen, human leukemia T cell antigen-Gp37, human lung carcinoma antigen L20, human lung carcinoma antigen L6, human milk fat globule antigen, IgE, KS ¼ pan-carcinoma antigen, LEA, lung adenocarcinoma F3 antigen, malignant human lymphocyte antigen-APO-1, melanoma antigen gp75, melanoma-associated antigen p97, neoglycoprotein, nuC242, polymorphic epithelial mucin antigen, prostate specific antigen, prostate specific membrane antigen, prostatic acid phosphate, SK-1 antigen, TAG-72, T-antigen, tumor antigen CA125, tumor antigen MUC1, tumor-specific transplantation type of cell-surface antigen, vascular endothelial growth factor, vascular endothelial growth factor-receptor, and αvβ3).

A suitable adjuvant, such as aluminum hydroxide or aluminum phosphate, preferably triggers, enhances, or prolongs an immune response to the cancer antigen. Additional materials, such as cytokines, chemokines, and bacterial nucleic acid sequences, like CpG, a toll-like receptor (TLR) 9 agonist as well as additional agonists for TLR 2, TLR 4, TLR 5, TLR 7, TLR 8, TLR9, including lipoprotein, LPS, monophosphoryl lipid A, lipoteichoic acid, imiquimod, resiquimod, and in addition retinoic acid-inducible gene I (RIG-I) agonists such as poly I:C, used separately or in combination with the described compositions, are also suitable adjuvants. Such antigens and adjuvants may be provided as a pharmaceutically acceptable salt, where appropriate.

As used herein, the term “an effective amount” of an ingredient (e.g., a molecule, compound, drug, or other agent), or of a pharmaceutical composition or formulation, refers to an amount sufficient to effect beneficial or desired results including, without limitation, clinical results such as decreasing symptoms resulting from the disease attenuating a symptom of cancer (e.g., the proliferation of cancer cells, tumor presence, tumor metastases, etc.), thereby increasing the quality of life of those suffering from the disease, decreasing the dose of other medications required to treat the disease, enhancing the effect of another medication such as via targeting and/or internalization, delaying the progression of the disease, and/or prolonging survival of individuals. When applied to a single active ingredient, the term refers to that ingredient alone. When applied to a combination, the term refers to combined amounts of the active ingredients that result in the therapeutic effect, whether administered in combination, serially, or simultaneously. An effective amount can be administered in one or more administrations and/or in one or more formulations or compositions. While individual needs vary, determination of optimal dosage ranges of effective amounts of an ingredient or composition is within the skill of the art. Empirical considerations, such as the biological half-life, also generally contribute to the determination of the dosage.

As used herein, the term “agent” refers to a biological, pharmaceutical, or chemical compound. Non-limiting examples include simple or complex organic or inorganic molecules, a polypeptide or peptide, a protein, an oligonucleotide, an antibody, an antibody derivative or fragment, a vitamin derivative, a carbohydrate, a toxin, or a chemotherapeutic compound. Agents that are employed in the disclosed methods and compositions can be randomly selected or rationally selected or designed.

The terms “polypeptide,” “oligopeptide,” “peptide” and “protein” are used interchangeably herein to refer to polymers of amino acids of any length, but especially lengths greater than 5, 10, 15, 20, or 25 amino acid residues. The polymer may be linear or branched, it may comprise modified amino acids, and it may be interrupted by non-amino acids. The terms also encompass an amino acid polymer that has been modified naturally or by intervention.

As used herein, the term “toxin” refers to any substance which affects an adverse response within a cell. For example, a toxin directed to a cancerous cell would have an adverse, sometimes deleterious effect, on the cancerous cell. Examples of toxins include, but are not limited to, a taxane, a maytansinoid, an auristatin, a calicheamicin, an anthracycline, a CC-1065 analog, docetaxel, cathepsin B or E, ricin, gelonin, Pseudomonas exotoxin, diphtheria toxin, and RNase; and radiolabeled molecules or antibodies.

Pharmaceutical Compositions

Where more than one ingredient or therapeutic agent is to be administered, the agents may be formulated together in the same formulation or composition, or may be formulated into separate compositions. Accordingly, in some embodiments, a cancer antigen and an adjuvant are formulated together in the same pharmaceutical composition. In alternative embodiments, a cancer antigen is formulated in one formulation or pharmaceutical composition, and an adjuvant is formulated in another separate formulation or pharmaceutical composition.

The pharmaceutical compositions of the present disclosure may include auxiliary ingredients or components such as pharmaceutically acceptable carriers, diluents and/or excipients. As used herein, the term “pharmaceutically acceptable” means approved by a regulatory agency of the Federal or a state government or listed in the U.S. Pharmacopeia or other generally recognized pharmacopeia for use in animals, and more particularly in humans. Such auxiliary ingredients are selected to not affect the biological activity of the active ingredients or agents. Such auxiliary ingredients are well known in the art and are relatively inert substances that facilitate administration of a pharmacologically effective substance or which facilitate processing of the active compounds into preparations that can be used pharmaceutically for delivery to the site of action.

In addition, the pharmaceutical composition or formulation may also include other carriers, or non-toxic, nontherapeutic, non-immunogenic stabilizers and the like. Pharmaceutically acceptable carriers include any and all suitable solvents, dispersion media, coatings, antibacterial and antifungal agents, absorption delaying agents, and the like that are physiologically compatible with a compound of the present disclosure. Examples of suitable aqueous and nonaqueous carriers which may be employed in the pharmaceutical compositions of the present disclosure include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. The composition, if desired, can also contain a minor amount of a wetting or emulsifying agent, or a pH buffering agent.

Pharmaceutical compositions of the present disclosure may also comprise pharmaceutically acceptable antioxidants for instance (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. Pharmaceutical compositions of the present disclosure may also comprise isotonicity agents, such as sugars, polyalcohols, such as mannitol, sorbitol, glycerol, or sodium chloride in the compositions. Prolonged absorption of the compositions may be brought about by including in the composition an agent that delays absorption, for example, monostearate salts and gelatin.

The compositions of the disclosure can be formulated as neutral or salt forms. Pharmaceutically acceptable salts include, but are not limited to those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

A variety of delivery systems and administration routes for providing the cancer antigen and/or adjuvant are available. Delivery systems that can be used to administer or deliver a cancer antigen and/or an adjuvant include, but are not limited to, encapsulation in liposomes, micelles, nanoparticles, microparticles, microcapsules, or other suitable carriers. Further, the molecules and/or compositions utilized in accordance with disclosed embodiments can be delivered in a controlled-release or sustained-release system. Techniques known in the art to produce sustained-release formulations comprising one or more of the subject molecules may be utilized.

In some embodiments, polymeric materials can be used to achieve delivery and/or controlled-release of the molecules. Examples of suitable polymers include, but are not limited to, poly(2-hydroxyethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), polyorthoesters, chitosan, and hyaluronic acid. A controlled-release system can be implanted, injected, or placed in proximity of or within the therapeutic target (e.g., lymph node).

In one embodiment, the compounds may be formulated to ensure proper distribution and efficacy in vivo. Pharmaceutically acceptable carriers for parenteral administration include sterile aqueous solutions or dispersions and sterile powders for the extemporaneous preparation of sterile injectable solutions or dispersion. Sterile solutions may be prepared by incorporating the active compound in the required amount in an appropriate solvent with one or a combination of ingredients, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof

Dosage and Administration

Dosage regimens in the above methods of treatment and uses are adjusted to provide the optimum desired response (e.g., a therapeutic response). For example, a single bolus (e.g., injected into a single lymph node of a subject) may be administered, or several divided doses may be administered (e.g., via injection into two or more lymph nodes of a subject). Pharmaceutical compositions may be formulated in dosage unit form for ease of administration and uniformity of dosage. Dosage unit form as used herein refers to physically discrete units suited as unitary dosages for the subjects to be treated; each unit contains a predetermined quantity of active compound or ingredient calculated to produce the desired therapeutic effect in association with any required pharmaceutical carrier. The specification for the dosage unit forms of the present disclosure are dictated by and dependent on (a) the characteristics of the active compound and the particular therapeutic effect to be achieved, and (b) any limitations in the art of compounding such an active compound for the treatment of sensitivity in individuals.

A physician skilled in the art may readily determine and prescribe the therapeutically effective amount of the ingredients and/or pharmaceutical composition required for a particular subject. The selected and effective dosage level will depend upon a variety of pharmacokinetic factors including the activity of the particular compositions of the present disclosure employed, the route of administration, the rate of excretion or release of the particular compound being employed, the duration of the treatment, other drugs, compounds and/or materials used in combination with the particular compositions employed, the age, sex, weight, condition, general health and prior medical history of the patient being treated, and like factors well known to those skilled in the medical arts. It will be appreciated that the effective dosage of the subject molecules used for treatment may increase or decrease over the course of a particular treatment. For example, the calculated dosage for administration may be based on the patient's body weight at baseline. Alternatively, a fixed dosage of the subject molecules used for treatment may be administered to a subject regardless of patient body weight.

The pharmaceutical compositions of the present disclosure may be administered by any suitable route and mode. Preferably, the disclosed compositions or compounds are administered via direct injection into one or more lymph node(s) of a subject. In some embodiments, the introduction of a cancer antigen and adjuvant into the lymph node(s) of a subject is provided for the treatment, prophylaxis, and amelioration of one or more symptoms associated with cancer or other disease, by introducing directly into a lymph node(s) of the subject an effective amount of such a combination, or pharmaceutical composition(s) comprising the same. In some embodiments, the cancer antigen and adjuvant are introduced together directly into a single lymph node of the subject. In other embodiments, the cancer antigen and adjuvant are introduced together directly into two or more lymph nodes of the subject (e.g., with half or a portion of the total dosage introduced into one lymph node and other half or portion of the total dosage introduced into another lymph node of the subject). In other embodiments, the cancer antigen is introduced into one or more lymph node(s), and the adjuvant is introduced into another one or more lymph node(s) different from the lymph node(s) into which the cancer antigen is introduced.

The cancer antigen formulation in combination with the adjuvant formulation may be administered concurrently (e.g., wherein formulations containing the molecules are administered at the same time or within about 24 hours of each other) or sequentially (e.g., wherein a first formulation containing the cancer antigen or adjuvant is administered and, at a later time such as at least 24 hours or more after administration of the first formulation a second formulation containing the other of the cancer antigen or adjuvant is administered to the subject). As used herein, the term “combination” refers to the use of more than one therapeutic agent (e.g., a cancer antigen and an adjuvant). The use of the term “combination” does not restrict the order in which the therapeutic agents are administered to a subject.

Treatment of a subject with a therapeutically or prophylactically effective amount of a cancer antigen and/or adjuvant preferably includes a single treatment, but can also include a series of treatments. For example, a subject may be treated with formulations containing the cancer antigen and adjuvant once, or once a week, twice a week, once every two weeks, once every three weeks, once every four weeks, once every six weeks, once every two months, once a year, etc.

In some embodiments, the methods and compositions of the present disclosure provide for the use of a secondary or additional therapeutic agent in addition to therapeutically effective amounts of the cancer antigen and adjuvant, such as, for example, an additional anti-cancer agent. In some embodiments, a cancer antigen and adjuvant is administered to a subject in need thereof, in further combination with other therapies known to those skilled in the art for the treatment or prevention of cancer, including but not limited to, current standard and experimental chemotherapies, hormonal therapies, biological therapies, immunotherapies, radiation therapies, or surgery.

Diagnostic Assays and Kits

The disclosed methods of compositions may be used to identify the presence or absence of cancerous cells, or the level thereof, which are circulating in blood and/or present in tissue(s) of a subject. Such antigen may be an intact antigen, or a fragment thereof that retains the ability to be detected according to methods known in the art. For example, such detection may be affected by FACS analysis using standard methods commonly used in the art. In some embodiments, a molecule coupled to or bearing a detectable label is utilized, e.g., in methods for aiding diagnosis. Examples of labels that may be used include but are not limited to a radioactive agent, an enzyme, or a fluorophore. Techniques for detection of a labeled molecule are well known in the art and include enzyme linked immunosorbent assays (ELISAs), immunoprecipitations, immunofluorescence, enzyme immunoassay (EIA), radioimmunoassay (RIA), and Western blot analysis.

As used herein, the term “labeled,” with regard to a molecule or structure, is intended to encompass direct labeling of such molecule or structure by coupling thereto (i.e., physically linking) a detectable substance (e.g., a radioactive agent or a fluorophore), as well as indirect labeling of the molecule or structure by reactivity with a detectable substance. As used herein, methods for “aiding diagnosis” refers to methods that assist in making a clinical determination regarding the classification, or nature, of cancer, and may or may not be conclusive with respect to the definitive diagnosis.

A biological sample may be obtained to assess an immune response of a subject. The term “biological sample” encompasses a variety of sample types obtained from a subject and can be used in a diagnostic or monitoring assay. The definition encompasses saliva, blood, and other liquid samples of biological origin, solid tissue samples such as a biopsy specimen or tissue cultures or cells derived therefrom, and the progeny thereof, for example, cells obtained from a tissue sample collected from a subject diagnosed with or exhibiting symptoms of a cancer (e.g., melanoma or lymphoma). The definition also includes samples that have been manipulated in any way after their procurement, such as by treatment with reagents, solubilization, or enrichment for certain components, such as proteins or polynucleotides, or embedding in a semi-solid or solid matrix for sectioning purposes.

The present disclosure also encompasses kits for prophylaxis and/or treatment of a cancer. The kit can comprise a cancer antigen and an adjuvant agent in a single formulation, or in separate formulations for mixing together. The kit can comprise a biodegradable material for use in preparing a formulation for direct intra-LN administration. The kit can comprise a suitable buffer and pharmaceutically acceptable excipients, carriers, and the like. The components or ingredients can be included in separate containers, or a single container with separate compartments. The containers can include sterile components, vials, ampules, tubes, a bolus, etc. The kit may comprise a needle suitable for direct administration of a composition of this disclosure into one or more lymph nodes, and a syringe, and/or other components for use in lymph node injection. The kit can additionally comprise printed material, such as instructions, and/or an indication that the kit is for use in treating, for example, a subject diagnosed with or exhibiting symptoms of a cancer.

Intra-LN Vaccine Deposition and Treatment Strategies

As discussed above, LNs are key tissues that integrate immune signals to coordinate adaptive immune responses, and thus are important targets for vaccines. Directly controlling the signals and local environment in LNs enables potent and safe immunotherapies without cell isolation, engineering, and reinfusion. In accordance with disclosed embodiments, intra-LN injection of immune signal-loaded biomaterial depots is utilized to directly control cancer vaccine deposition. How the combination and geographic distribution of signals in and between LNs impact anti-tumor response was evaluated to guide specific vaccination and/or treatment strategies. As shown in healthy and diseased mouse models, the relative proximity of antigen and adjuvant to one another in LNs, and to tumors, define unique local and systemic characteristics of innate and adaptive response. Further, blocking immune cell egress diminishes immunity in contralateral LNs, revealing T cell migration patterns important in immunity. These factors controlled survival in mouse models of lymphoma and melanoma. With appropriate geographic signal distributions, a single intra-LN vaccine treatment conferred nearly complete survival to primary tumor challenge and to re-challenge 100 days later, without additional treatments. Thus, informed design criteria may be utilized for immunotherapy treatments that leverage biomaterials for loco-regional LN therapy. Thus, specific and targeted responses may be generated without systemically exposing subjects to potent or immunotoxic drugs.

Various embodiments of the disclosure are illustrated using PLGA as a representative biodegradable depot or carrier material. However, various other biodegradable materials, including but not necessarily limited to biodegradable polymers, may be utilized in accordance with the present disclosure, provided that the cancer antigen and/or the adjuvant can be exposed to immune cells in the lymph nodes concurrent with or subsequent to administration. For example, as an alternative to PLGA, the biodegradable material can comprise poly(glycolide) (PGA), poly(L-lactide) (PLA), or poly(beta-amino esters). In some embodiments, the biodegradable material may be a hydrogel, an alginate, or a collagen. In some embodiments, the biodegradable material can comprise a polyester, a polyamide, or polyethylene glycol (PEG). The cancer antigen and/or adjuvant may be coupled to or encapsulated with the biodegradable material. In some embodiments, the antigen and/or the adjuvant are released over a period of time, such as in an extended-release formulation where the components are released on the time scale of hours, days, or weeks. The carriers or depots are preferably microparticles having a size sufficiently large such that diffusion from LN(s) is limited or restricted, e.g., having a diameter of between about 1-100 μm, preferably between about 1-10 μm, more preferably between about 2-5 μm.

Additional characteristics and features of the present disclosure will be further understood through reference to the following additional examples and discussion, which are provided by way of further illustration and are not intended to be limiting of the present disclosure.

In accordance with the present disclosure, geographic delivery of simple biomaterial systems is utilized to evaluate how the relative proximity of antigen and adjuvant loaded in polymer depots impacts injected LNs and untreated LNs, and how communication between these sites determines efficacy in common pre-clinical cancer models. In particular, we used intra-LN injection to deliver widely-used signals with broad literature relevance to inguinal LNs of mice: ovalbumin (OVA) as an antigen, a molecular TLR agonist (polyinosinic:polycytidylic acid, referred to herein as “Poly(I:C)” or “PolyIC”) as an adjuvant, and poly(lactic-co-glycolic acid) microparticle depots (PLGA MPs) as carriers (Abeyrathne, E. D. N. S. et al., Egg white proteins and their potential use in food processing or as nutraceutical and pharmaceutical agents—A review. Poultry Science 92:3292-3299 (2013); Caskey, M. et al. Synthetic double-stranded RNA induces innate immune responses similar to a live viral vaccine in humans. Journal of Experimental Medicine 208:2357-2366 (2011); Antonelli, L. R. V. et al. Intranasal Poly-IC treatment exacerbates tuberculosis in mice through the pulmonary recruitment of a pathogen-permissive monocyte/macrophage population. J Clin Invest 120:1674-1682 (2010)).

The disclosed platform mimics a key goal of robust adjuvant systems: adjuvant particles ready to be mixed with tumor antigen(s) or subject-specific lysates. As shown by the data, deposition of dose-matched treatments to a single LN, split equally in two LNs, or segregated with antigen and adjuvant in separate LNs resulted in vastly different local and systemic immunological responses. These changes include distinct innate immune cell activation profiles and antigen-specific T cell responses in LNs and blood. These geographic parameters led to large distinct efficacies during melanoma and lymphoma models, with a single treatment of the most potent signal distribution achieving near-complete efficacy during challenge and re-challenge over a 100 day period.

Materials and Methods

Microparticle Synthesis. Degradable MPs were synthesized via a double-emulsion, solvent evaporation technique as previously described (Ribas, A. et al. Intra-lymph node prime-boost vaccination against Melan A and tyrosinase for the treatment of metastatic melanoma: results of a phase 1 clinical trial. Clin Cancer Res 17:2987-2996 (2011); G. Senti et al., Intralymphatic allergen administration renders specific immunotherapy faster and safer: a randomized controlled trial. PNAS 105(46):17908-17912 (2008); G. Senti et al., Intralymphatic immunotherapy for cat allergy induces tolerance after only 3 injections. J Allergy Clin Immunol 129(5):1290-1296 (2012)).

Briefly, to form lipid stabilized particles, 1,2-dioleoyl-sn-glycero-3-phosphocholine, 1,2-distearoyl-sn-glycero-3-phosphoethanolamine-N-[amino(poly ethylene glycol)-2000], and 1,2-dioleoyl-3-trimethylammonium propane (Avanti Polar Lipids) were prepared at a 60:20:20 mol ratio and dried under nitrogen. For each particle batch, 80 mg of PLGA (Sigma) was dissolved with 5.15 μmol of lipids in 5 mL of dichloromethane. An inner aqueous phase containing 500 μL of water or 5 mg of PolyIC (Invivogen) in 500 μL of water was added to the organic phase containing polymer and lipid and sonicated for 30 seconds at 12 W to form the first emulsion. This emulsion was then added to 40 mL of water, homogenized for 3 min. at 16,000 rpm, and allowed to evaporate overnight while stirring to remove any excess organic solvent. After overnight stirring, particles were passed through a 40 μm cell strainer to remove any large aggregates and collected via centrifugation (5000 g, 5 min, 4° C.). Supernatants were removed and particles were washed three times with 1 mL of water then suspended in water or PBS for animal studies, or lyophilized and stored at 4° C. prior to use. For the preparation of fluorescently-labeled particles, 10 μL of DiO (Invitrogen) was added to the organic phase prior to forming the emulsion.

Microparticle Characterization and Cargo Release. Particle diameter was determined using an LA-950 laser diffraction analyzer (Horiba). Zeta potential was measured using a Malvern Zetasizer Nano Z590. The loading level of PolyIC was determined via UV/Vis spectrophotometry after hydrolyzing a known mass of lyophilized PolyIC MPs overnight in 0.2M NaOH. Absorbance values were compared to standard curves of known masses of PolyIC to determine the mass of cargo per mass of polymer. MPs were imaged using a Hitachi SU-70 Schottky field emission gun scanning electron microscope after sputter coating lyophilized particles with gold. Cumulative release of PolyIC from MPs was characterized by suspending a known mass of MPs in media (RPMI 1640) and incubating at 37° C. At given intervals, media was removed and replaced with fresh media. The removed media was analyzed via UV/Vis spectrophotometry and absorbance values were compared to standard curves of known masses of PolyIC in media.

Mouse Preparation and intra-LN Vaccination. For each animal study, a small region of fur was removed from the lateral hind quarter of 6-10 week old C57BL6 mice (The Jackson Laboratory) by shaving the area and applying a mild depilatory. Tracer dye (1% w/v Evan's Blue) was injected subcutaneously on each side of the tail base as previously reported (Ribas, A. et al. Intra-lymph node prime-boost vaccination against Melan A and tyrosinase for the treatment of metastatic melanoma: results of a phase 1 clinical trial. Clin Cancer Res 17:2987-2996 (2011); G. Senti et al., Intralymphatic allergen administration renders specific immunotherapy faster and safer: a randomized controlled trial. PNAS 105(46):17908-17912 (2008); G. Senti et al., Intralymphatic immunotherapy for cat allergy induces tolerance after only 3 injections. J Allergy Clin Immunol 129(5):1290-1296 (2012); Johansen, P. et al. Direct intralymphatic injection of peptide vaccines enhances immunogenicity. Eur J Immunol 35:568-574 (2005)). After allowing for the tracer dye to drain to the inguinal LNs for visualization (approximately 16 hours), a 31G insulin needle was used to inject 10 μL containing the indicated treatment into inguinal LNs.

To test how the localization of vaccine cargos to LNs impacted the local and systemic response MPs containing adjuvant (PolyIC) were mixed with soluble antigen (ovalbumin, OVA, Worthington) and introduced via intra-LN injection into one LN (‘One LN’), two LNs (Two LN’), or were split with the adjuvant MPs in one LN and the antigen in another LN (‘Split LN’). Within these studies, the overall dose of vaccine (adjuvant and antigen) delivered to the mouse was fixed while the dose delivered to an individual LN varied between treatments. For example, in the One LN treatment, 1 mg of PolyIC MPs containing ˜9 μg of PolyIC was co-injected with 50 μg of OVA suspended in 1×PBS to the left inguinal LN of a mouse. The right inguinal LN of the same mouse received only 1×PBS as an injection. In that same study, for a Two LN treatment, 0.5 mg of PolyIC MPs containing ˜4.5 μg of PolyIC was co-injected with 25 μg of OVA suspended in 1×PBS to the left and right inguinal LN of a mouse. In the Split LN treatment, 1 mg of PolyIC MPs containing ˜9 μg of PolyIC was injected into the left inguinal LN while 50 μg of OVA suspended in 1×PBS was delivered into the right inguinal LN. All studies involving mice were carried out in compliance with federal, state, and local laws and followed institutional guidelines, including the Guide for the Care and Use of Laboratory Animals and the Animal Welfare Act. All experiments were reviewed and approved by the University of Maryland's Institutional Animal Care and Use Committee (IACUC).

LN and Immune Cargo Imaging. For immune cargo and LN imaging, PolyIC was labeled with Cy5 per the manufacturer's protocol (Mims Bio) and was encapsulated in MPs. FITC OVA (Thermofisher) was also used. 24 hours after intra-LN injection as described above, inguinal LNs were removed and frozen in OCT compound (Tissue-Tek). Frozen tissue was then sectioned at 6 μm thickness, air-dried for 2 hours, fixed in acetone, and washed in 1×PBS. LN sections were visualized using an Olympus IX83 fluorescent microscope and processed using ImageJ by comparison to an antibody isotype control. All images were equally adjusted across similar channels. Imaging of live and dissected whole mice was performed using a Perkin-Elmer IVIS Spectrum in vivo imaging system. Intra-LN injections were performed as described above with FITC-labeled OVA and PLGA MPs synthesized with Cy5-labeled PolyIC. Animals were imaged live at 4 hours after injection then euthanized and dissected to image. Image analysis was performed using Living Image and quantitative measures were performed using region of interest (ROI) analysis of total radiant efficiency.

Innate Cell Microparticle Uptake and Activation. To determine which innate cell subtypes were responsible for particle uptake, 1 mg of MPs labeled with DiO were introduced into the LNs of mice via intra-LN injection with either a One LN, Two LN, or Split LN treatment. 24 hours after treatment, mice were euthanized, the inguinal LNs were removed, and placed in 1×PBS. Tissues were processed into single-cell suspensions by mechanical dissociation through a 40 μm strainer. After washing once with 100 μl of FACS buffer (1×PBS with 1% w/v bovine serum albumin, Sigma), cells were collected via centrifugation and blocked with Fc Block (anti-CD16/CD32, BD) for 10 minutes at room temperature to inhibit any nonspecific binding. Cells were then incubated for 30 minutes at room temperature with antibodies against cell surface markers including CD11b, CD8, F4/80, Ly6G, B220, and CD11c (all antibodies were from Becton Dickinson (BD) or Biolegend unless otherwise mentioned). Cells were then washed twice, suspended in FACS buffer, and particle uptake was quantified via flow cytometry (BD FACSCanto II) and analyzed using Flowjo (v.10, Treestar). To determine innate cell activation, mice were treated as above with PolyIC MP and OVA via intra-LN injection with either a One LN, Two LN, or Split LN treatment. Seven days after treatment, inguinal LNs were removed and stained against cell surface markers including CD11c, F4/80, CD40, CD80, CD86, I-A/I-E.

Antigen-Specific CD8⁺ T cell Quantification via Tetramer Staining in Blood and Chemokine Expression. At the indicated times after treatment of mouse LNs as above (e.g., Day 0, 7, 14, 21, 28), approximately 100 μL of blood was collected from anesthetized mice via submandibular bleeding. Red blood cells were removed by treating with 1 mL ACK lysis buffer (Lonza) for 5 minutes at room temperature. After pelleting cells by centrifugation (800×g for 5 minutes at 4° C.) and aspirating the supernatant, cells were again treated with ACK lysis buffer, centrifuged, and the supernatant was removed. Following a 1 mL wash with FACS buffer, cells were blocked using Fc Block (anti-CD16/CD32, BD) for 10 minutes at room temperature to inhibit any non-specific binding. The cells were then stained with anti-SIINFEKL tetramer (H-2 Kb OVA, MBL International) for 30 minutes at room temperature followed by staining with antibodies for surface markers including CD4 and CD8 for 20 minutes at room temperature. After staining, cells were washed twice with 200 μL of FACS buffer, suspended in FACS buffer containing DAPI, and transferred to 5 mL flow cytometry tubes before being quantified on a cell analyzer (BD, FACSCANTO ITC). The percentage of antigen-specific cytotoxic T cells (DAPI⁻, CD8⁺, tetramer⁺) were quantified. For experiments to determine chemokine expression, cells were prepared as above and stained with antibodies for CCR7 and CCR5. Chemokine expression was evaluated as the overall number of positive cells or the percentage under CD8⁺ cells and CD8⁺, Tet⁺ cells.

Antigen Specificity and Lymphocyte Enumeration in LNs. At indicated terminal time points (e.g., Day 7 or 28 post-vaccination), mice were euthanized, inguinal LNs were collected, placed in PBS, and processed into single-cell suspensions by mechanical dissociation through a 40 μm strainer. Cells were washed once with 100 μL of FACS buffer then blocked with Fc Block for 10 minutes at room temperature. Cells were stained for lymphocyte populations and antigen-specific tetramer levels. First, 25 μL of anti-SIINFEKL tetramer was added and incubated for 30 min at room temperature followed by addition of 25 μL of antibodies against surface markers including CD4 and CD8 and incubated for 20 min at room temperature. Cells were then washed and evaluated, as above. The frequency of each cell population (percent of parent population) and number of counted cells per identical acquisition volume (80 μL) was evaluated.

Investigation of Adjuvant-Involvement on Split LN Treatments and Effect of Inhibition of Lymphocyte Trafficking on Antigen Specificity. To determine the influence of adjuvant (PolyIC) delivery in the Split LN treatment group on antigen-specificity as quantified by SIINFEKL tetramer, mice were treated as described above for Split LN treatment with soluble OVA and either PolyIC MPs or with PBS. Antigen-specific CD8⁺ T cells in the blood and LNs were quantified using SIINFEKL tetramer as described above. Antigen-specific T cells were reported as a percentage of Live, CD8⁺ cells. In studies to determine the effect of lymphocyte trafficking on local and systemic antigen-specificity, FTY720 (Sigma) was administered daily at 1 mg/kg via intraperitoneal injection one day prior to vaccination until the end of the experiment (7 days after vaccination). Following vaccination, antigen-specific CD8⁺ T cells in the blood and LNs were quantified using SIINFEKL tetramer as described above and reported as a percentage of Live, CD8⁺ cells and an overall number of cells from an identical acquisition volume (80 μL).

Tumor Inoculation, Quantification, and Rechallenge and Therapeutic Tumor Studies. In preventative tumor studies, 7 days after treating mice with the indicated vaccines, mice were administered either 500,000 B16-OVA cells (ATCC) or 1,000,000 E.G7-OVA cells (ATCC) in 100 μL of 1×PBS subcutaneously at the hind flank. Each day following inoculation, body weight was monitored and tumor burden was calculated as a product of two orthogonal diameters. Mice were euthanized according to IACUC-approved humane endpoints when the aggregate tumor burden exceeded 150 mm². In the B16-OVA studies, mice that either did not establish or were able to clear the initial tumor were rechallenged with 100,000 B16-OVA cells 13 weeks after initial tumor inoculation. The percentage of mice that either did not establish or cleared the secondary tumor were quantified as a percentage of the mice surviving the initial inoculation. In therapeutic tumor studies, mice were first inoculated with 500,000 B16-OVA cells (ATCC) in 100 μL of 1×PBS subcutaneously at the hind flank. Then, 10 days after inoculation when tumors were palpable, mice were randomized into groups and treated with indicated vaccines either via intra-LN into the tdLN or subcutaneous injection at the tail base on the tumor draining side. Mouse weights and tumor burdens were monitored as above with identical endpoints.

Statistical Analysis. No pre-processing of data (transformation or removal of outliers) was performed prior to statistical analysis. Sample sizes for each analysis are indicated in figure legends. Student's t tests were used in comparison of two groups. One-way ANOVA with a Tukey post-test was used to compare three or more groups, or two-way ANOVA for comparisons over time. In all cases, analyses were carried out with GRAPHPAD PRISM® (version 8.4.3). Multiple comparisons of survival curve analysis were performed in Microsoft Excel as explained in GRAPHPAD PRISM® using the Bonferroni method and the logrank p-values. Error bars in all panels represent the mean±SEM and p-values ≤0.05 were considered significant with levels of significance were defined as *p<0.05; **p<0.01; ***p<0.001; ****p<0.0001 unless otherwise noted. An absence of a symbol denotes non-significant findings and all statistical comparisons depicted are versus Sham unless otherwise noted.

Results and Discussion

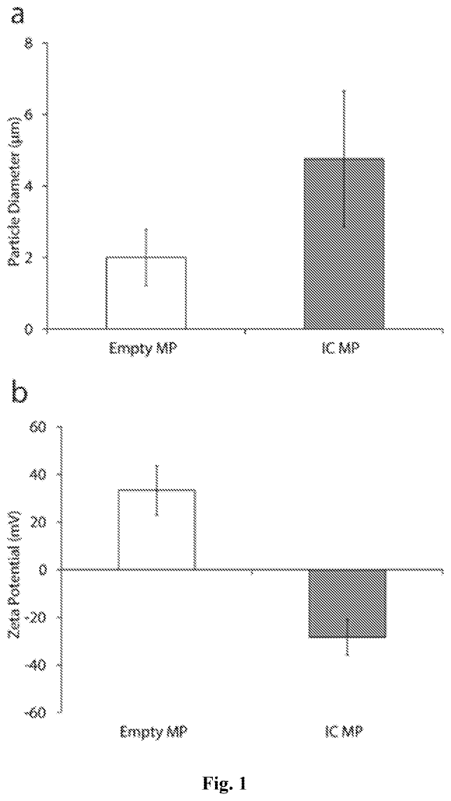

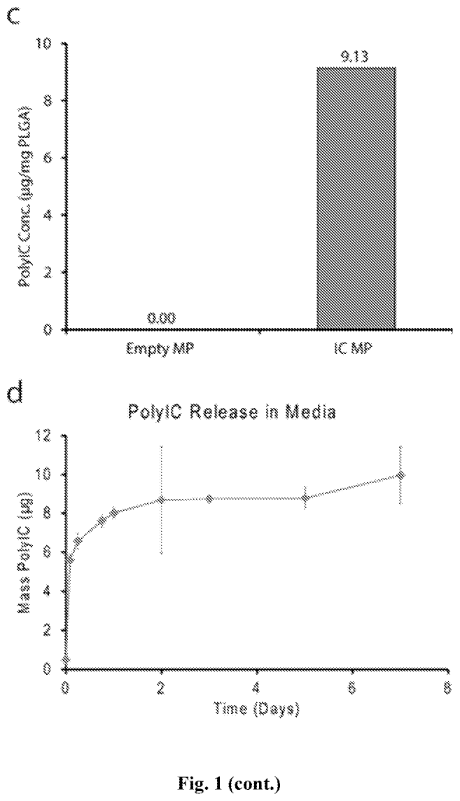

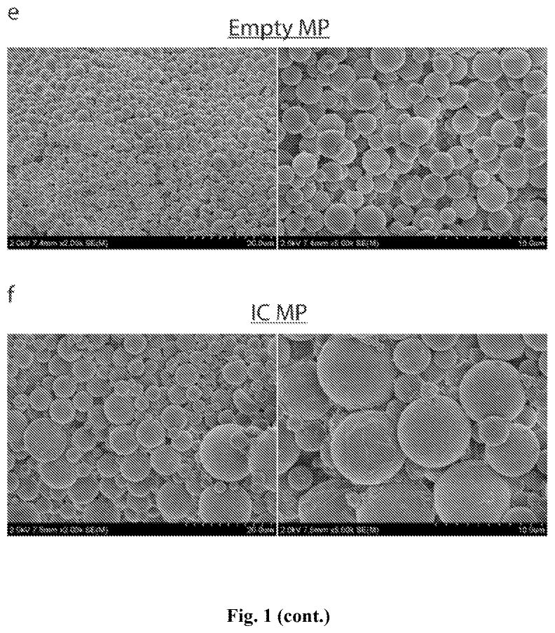

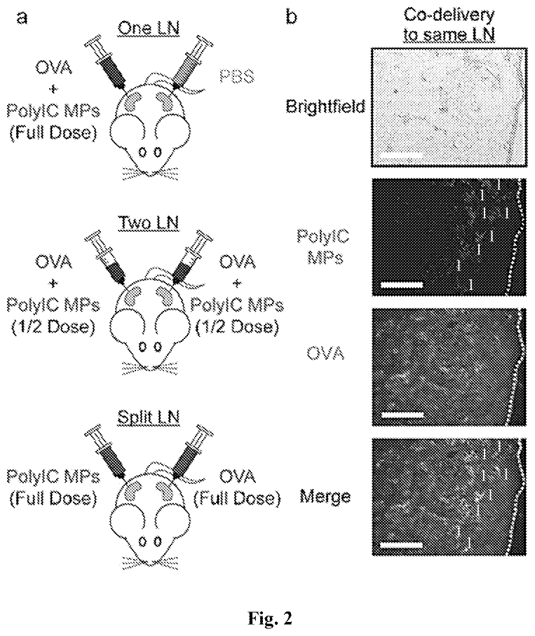

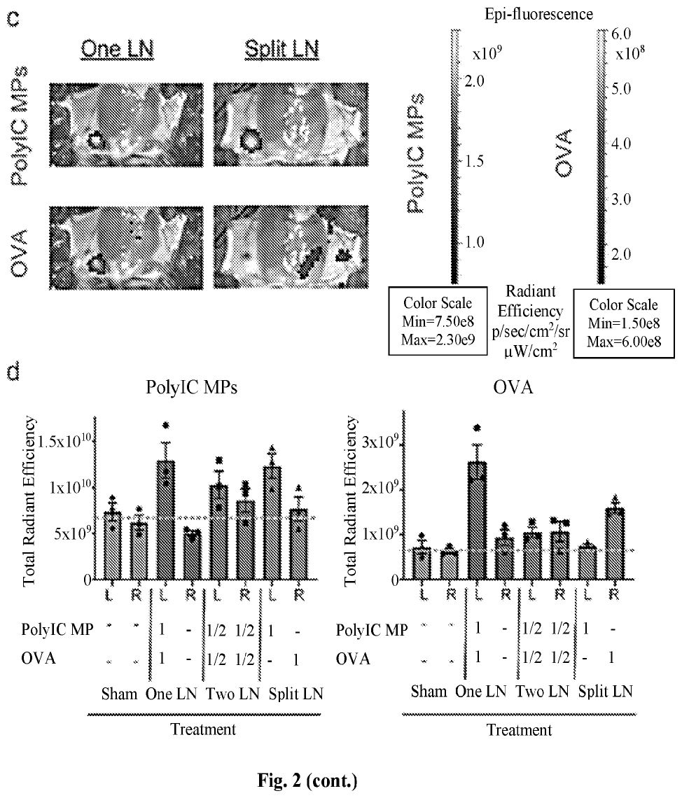

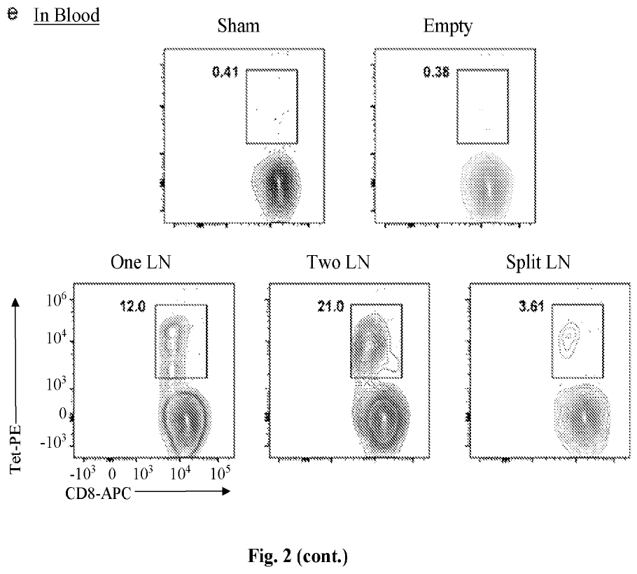

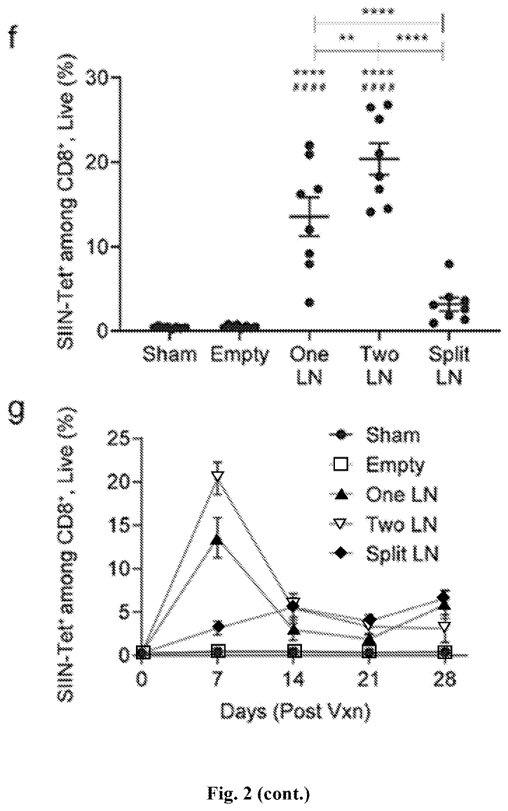

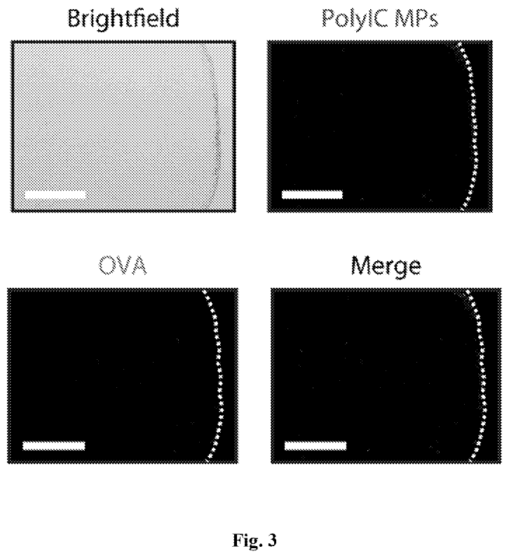

Relative location of antigen and adjuvant loaded MPs determine distinct magnitudes of systemic response. We first used intra-LN delivery to confirm that the spatial juxtaposition of antigen and adjuvant that the immune system relies on can be selectively directed using local delivery of intra-LN depots. These particles were synthesized to be too large to drain from LNs, and thus diffusion limited, e.g., having an average diameter of between about 1-10 μm, preferably between about 2-5 μm (see, e.g., Jewell, C. M. et al., In situ engineering of the lymph node microenvironment via intranodal injection of adjuvant-releasing polymer particles. Proc. Natl. Acad. Sci. U.S.A. 108:15745-15750 (2011)). MPs exhibited characteristic cargo loading, size, surface charge, morphology, and release kinetics (FIG. 1). Mice were treated by administering a fixed total dose of OVA mixed with PolyIC MPs to either i) a single inguinal LN (“One LN”), ii) two inguinal LNs each receiving half the total dose (“Two LN”), or iii) two inguinal LNs, with one LN receiving all of the antigen and one LN receiving all of the PolyIC MPs (“Split LN”) (FIG. 2a). To determine if immune signals are retained in treated LNs, mice were treated with both fluorescently-labeled PolyIC MPs and OVA. 24 hours later, histological analysis of treated LNs revealed deposition of both PolyIC MP (red) and OVA (green) into distinct LNs (FIG. 2b) compared to Sham treated LNs (FIG. 3). Of note, the soluble OVA was more diffuse than the PolyIC, which was encapsulated in MPs.

Quantitative studies were subsequently performed using IVIS imaging in which mice were treated as in FIG. 2a with “One LN”, “Two LN”, or “Split LN” strategies. The geographic distribution of signals was then visualized after 4 hours (FIG. 2c). Similar to the histological results in FIG. 2b, images in FIG. 2c revealed localization to the expected LNs (e.g., “One LN”, “Split LN”) with more diffuse signal for soluble OVA. Quantification of each cohort using 3D image voxel analysis (FIG. 2d) revealed cargo in LNs at relative levels corresponding to the treatment scheme. For example, “Two LN” treatment of LNs where each node received half a total dose of immune cue exhibited approximately half of the signal of a node treated with a full dose.

To investigate the influence of this geographic distribution on antigen-specific, adaptive immune response, blood was collected from mice 7 days after vaccination and MHC-I tetramer (SIINFEKL epitope, SIIN) was used to quantify the frequency of OVA-specific CD8⁺ T cells recognizing the SIINFEKL epitope fragment contained in OVA (FIG. 2e). Treating two LNs—each LN with half the total dose—resulted in the greatest frequency of OVA-specific CD8⁺ T cells after 7 days, with extremely strong responses indicated by an average frequency of 20.4%. Administration of the total dose into a single LN resulted in very strong (13.6%), but significantly lower responses compared to “Two LN” treatment (FIG. 2f). In contrast, the “Split LN” regimen generated a dramatically lower frequency 7 days after injection (3.16%) (FIG. 2f), confirming the importance of co-localizing antigen and adjuvant to generate potent immune responses. When evaluating the response over time, the largest differences between the percentage of SIIN-Tet⁺, CD8⁺ T cells were present 7 days after treatment (FIG. 2g). At 14 days post-injection, the “Two LN” (FIG. 2g, blue) and “One LN” (FIG. 2g, red) treatments contracted, whereas the “Split LN” treatment regimen resulted in a slow, modest increase (FIG. 2g, green). The latter indicates a low level of trafficking of adjuvant or antigen between nodes. Overall, the results of FIG. 2 demonstrate intra-LN depots effectively localize signals to LNs, allowing rational control of where signals are localized and integrated.

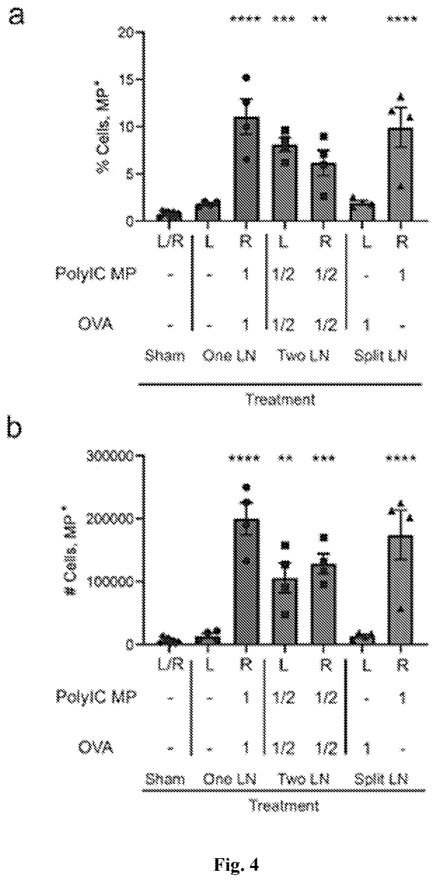

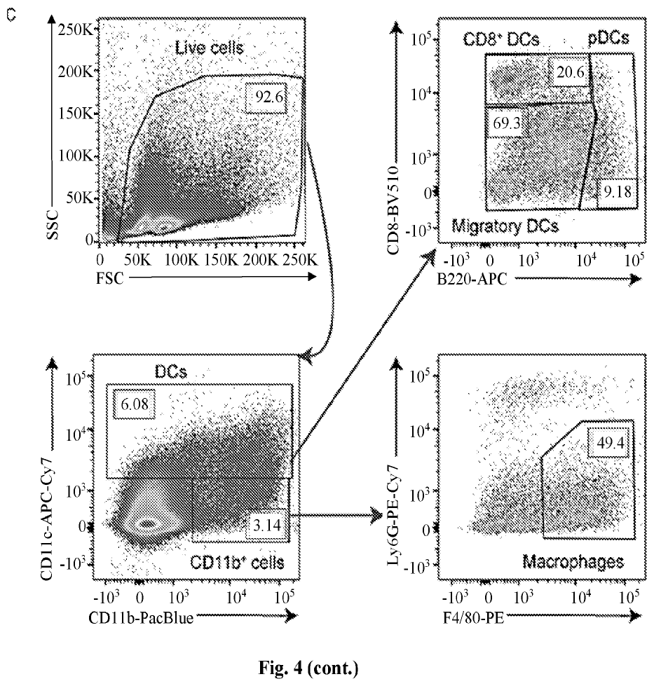

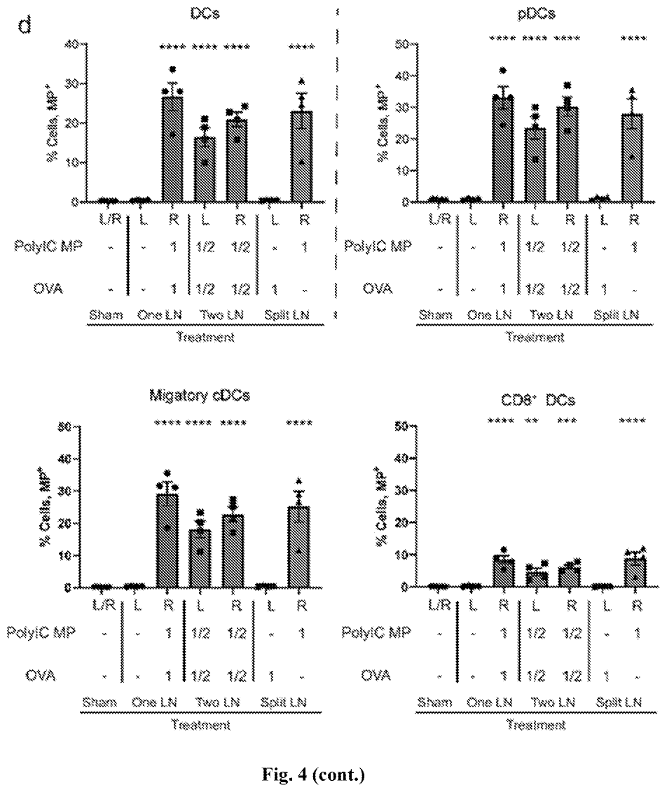

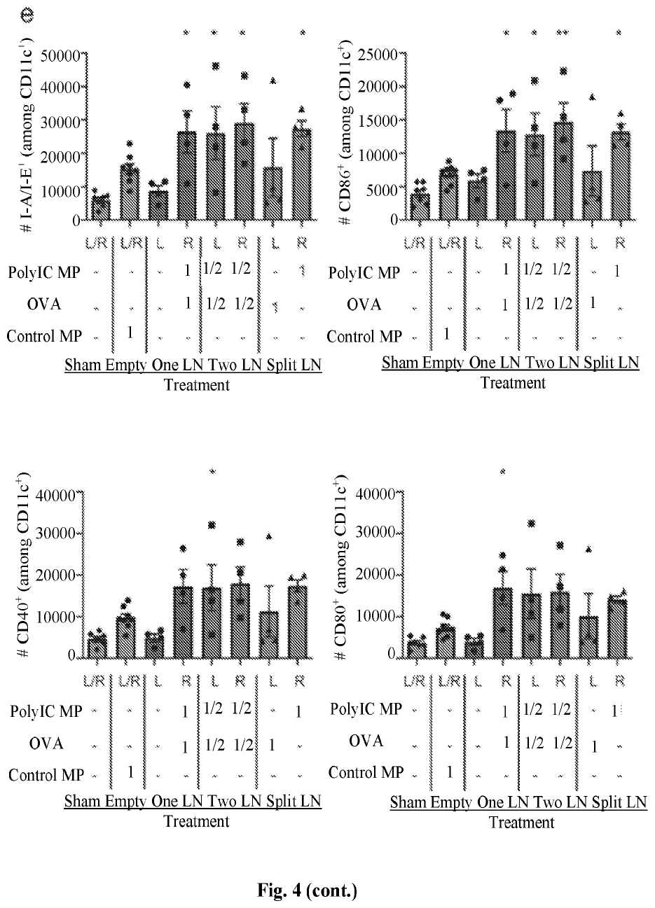

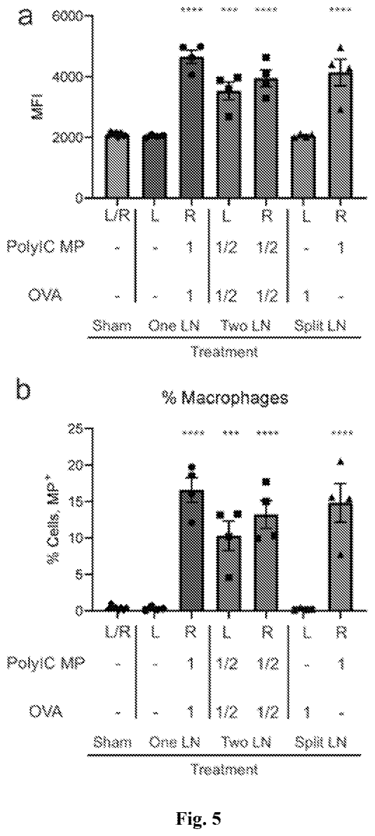

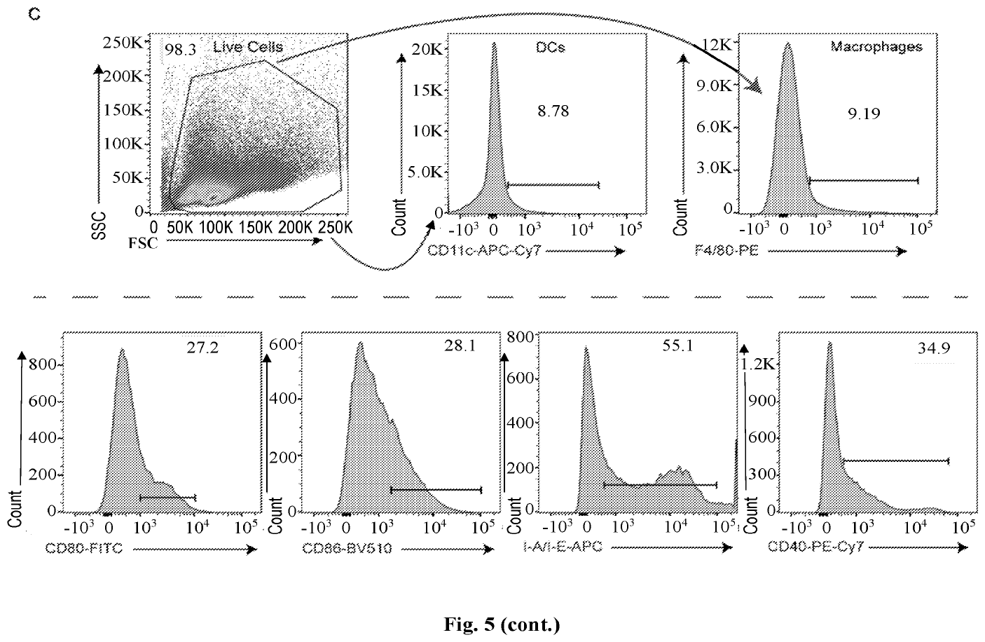

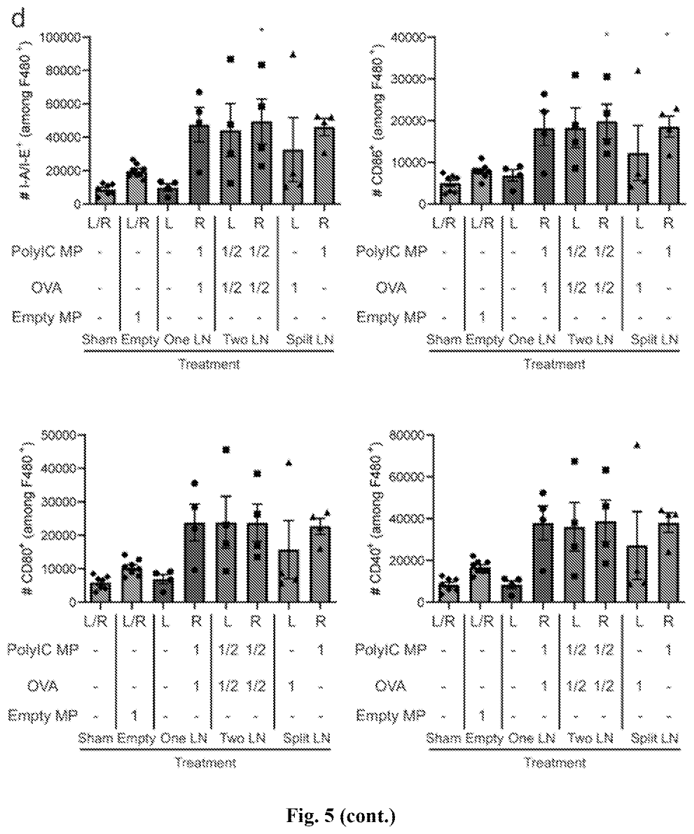

Particle uptake and activation correlates with geographic signal distribution in LNs. We next directly studied the degree to which depots spatially-restrict the effects of adjuvant; this is clinically important since many cancer immunotherapeutics used to generate anti-tumor responses are also toxic (L. B. Kennedy & A. K. S. Salama, A review of cancer immunotherapy toxicity. CA Cancer J Clin 70(2):86-104 (2020)). Mice were treated with OVA and fluorescent MPs using the “One LN,” “Two LN,” or “Split LN” regimens as above. When the total MP dose was administered to a single LN (i.e., “One LN” and right LN of “Split LN” regimen), approximately 10% of cells in the LN were positive for these large particles after 24 hours (FIG. 4a,b). In the case of the “Two LN” treatments, where half of the total dose of MPs was delivered to each LN, there was a corresponding decrease in the percentage of cells positive for particles by approximately half (FIG. 4a,b). No significant increases were observed in LNs of mice that were contralateral to an injected LN (FIG. 4a,b), further demonstrating the spatial control of this intra-LN injection approach. Assessment of the mean fluorescent intensity (MFI) also confirmed these biodistributions (FIG. 5a), further validating that the “mass balance” of the injected dose is almost complete, with a comparable number of cells taking up particles when integrating the portions of the total dose administered to each LN.

We next analyzed the MP uptake as a function of cell type, focusing on several important APC subsets: macrophages (CD11b⁺/CD11c⁻, F4/80⁺/Ly6G⁻) and dendritic cells (DCs; CD11c⁺), along with DC subsets of plasmacytoid DCs (CD11c⁺, B220⁺), CD8⁺ DCs (CD11c⁺, B220⁻/CD8⁺), and the remaining LN resident/migratory conventional DCs (CD11c⁺, B220⁻/CD8⁻); the gating scheme is shown in FIG. 4c. In LNs receiving particles, these studies revealed that 10-15% of macrophages and 20-25% of DCs were positive for MP signal (FIG. 4d, FIG. 5b). As with the data for uptake by the total population of cells (FIG. 4a,b), the level of uptake in the “Two LN” regimen (where each LN receives half the dose) was generally lower than in LNs receiving the total dose of MPs (FIG. 4d). When assessing specific DC subsets, all DCs phagocytosed MPs, with plasmacytoid DCs and conventional DCs exhibiting similar levels of uptake; CD8⁺ DCs internalized fewer MPs, but with a similar trend across treated groups (FIG. 4d). This diminished uptake by CD8⁺ DCs may be due in part to their specialized endocytic compartments which are optimized for cross-presentation of antigen (O. P. Joffre et al., Cross-presentation by dendritic cells. Nat Rev Immunol 12(8):557-569 (2012)).

Next, we used key surface activation markers to determine if APC activation correlated to uptake levels. Compared to a control injection of Empty MPs which modestly increased activation, all LNs receiving PolyIC MP, regardless of the specific injection scheme, exhibited further increased DC activation, as indicated by elevated I-A/I-E (mouse MHCII) expression and expression of costimulatory markers (CD86, CD80, and CD40) (FIG. 4e, FIG. 5c). Interestingly, activation was not observed in the Sham (PBS-treated) LNs, even when a full dose was injected contralaterally in the “One LN” regimen (FIG. 4e, left red bar); this underscores the precise spatial control in this platform. Analogous results were obtained when evaluating macrophages (FIG. 5d). In assessing the left LN of the “Split LN” regimen, which only received OVA, there was a slight trend toward increased activation in macrophages (FIG. 5d), and to a lesser extent, DCs (FIG. 4e). This suggests several possible explanations investigated further below, including drainage or transport of PolyIC from the right LN to the left LN, or migration of activated APCs between these sites.

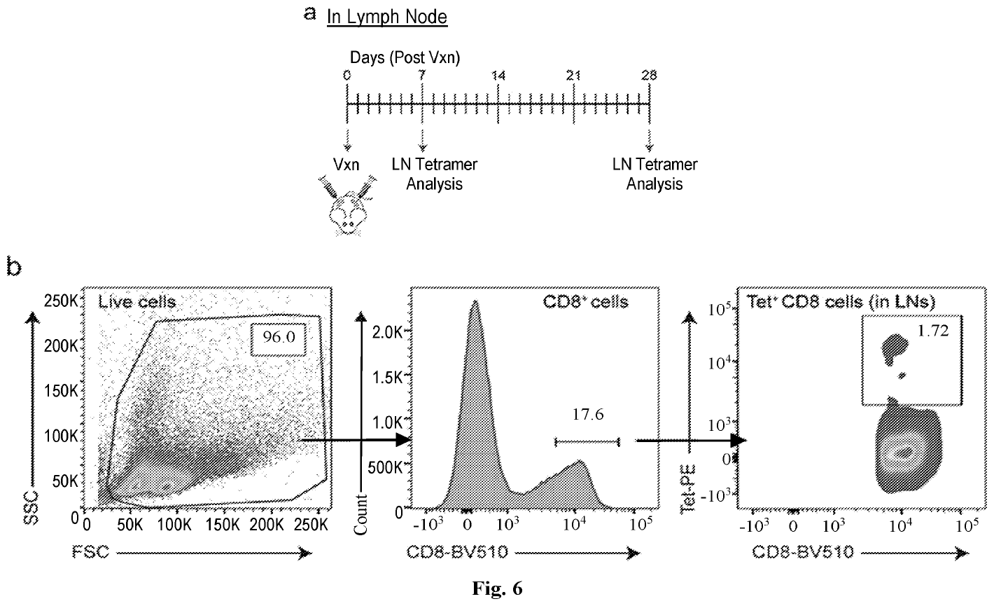

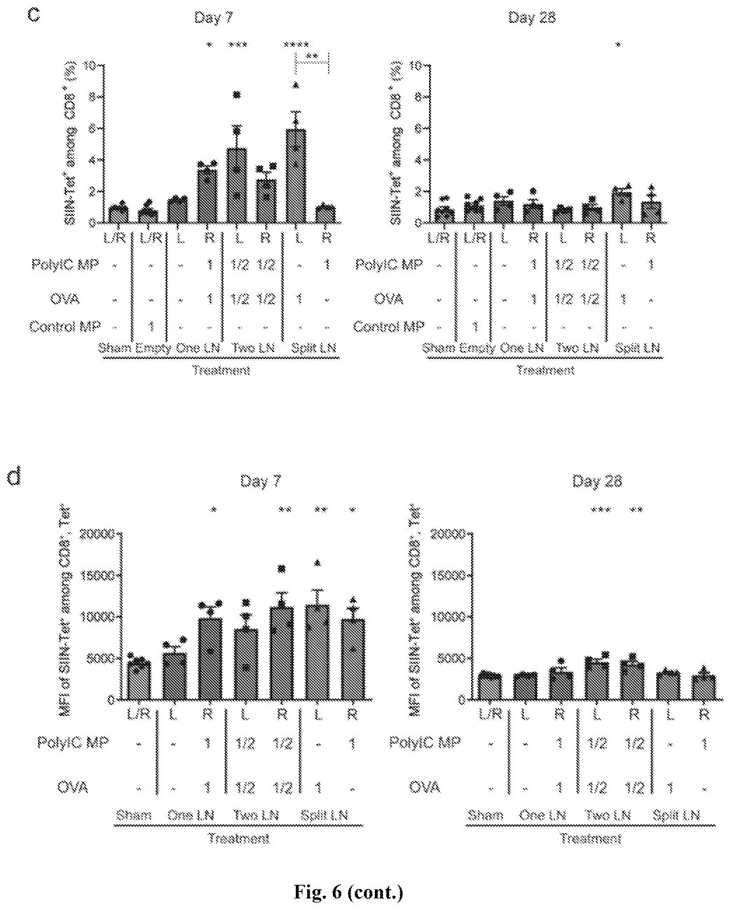

Depot and relative signal localization dictate local expansion of antigen-specific T cells. In FIG. 2, we measured systemic antigen-specific response as a function of signal distribution. FIG. 4 revealed there are also distinct local differences in APC activation within LNs. Thus, we next assessed the level of antigen-specific T cells present directly in LNs using the same geographic injection schemes from above. Following injection (Day 0), LNs were isolated after 7 and 28 days (FIG. 6a), then the frequency of SIIN-Tet⁺, CD8⁺ cells was determined in both treated and untreated LNs using the gating scheme in FIG. 6b. 7 days after treatment, at the peak of T cell expansion and systemic responses observed in FIG. 2g, only LNs that received OVA (i.e., “One LN”, right; “Two LN”, right and left; “Split LN”, left) exhibited increased levels of antigen-specific cells (FIG. 6c, left plot). Interestingly, analysis of the MFIs among the CD8⁺, Tet⁺ cells revealed that any LN that received an immune signal (antigen or adjuvant), exhibited increased signal (FIG. 6d, left plot). Of note, the left LN from the “Split LN” treatment regimen (which received only OVA), contained a similarly high level of antigen-specific CD8⁺ T cells to those that received both OVA and PolyIC MPs. However, the APCs in these nodes were not strongly activated (FIG. 2e) and this same “Split LN” scheme did not result is significant antigen-specific T cells outside of the LN (i.e., circulating in blood) (FIG. 2e-g). Along these same lines, even though APCs in the right LN during the “Split LN” regimen were strongly activated (FIG. 2e, FIG. 5d), the percentage of antigen-specific T cell levels in this LN were very low due to the absence of antigen (FIG. 6c, left plot). This also indicates antigen, even though delivered here in a soluble form and not formulated in a MP, is not able to move or be carried from the left LN to the right LN over the period of 7 days. At longer time points (i.e., day 28 after injection), the antigen-specific T cell levels in the LNs contracted for all cases (FIG. 6c,d, right plots). Together, these observations indicate that T cells may expand in the LN during the “Split LN” scheme due to the presence of cognate antigen, but that these cells are not activated and cannot exit the LN to blood as functional antigen-specific T cells. To test these hypotheses directly, we next studied how the presence or absence of adjuvant in the LN influences local and systemic antigen-specific responses to antigen introduced to a contralateral LN (i.e., “Split LN” regimen).

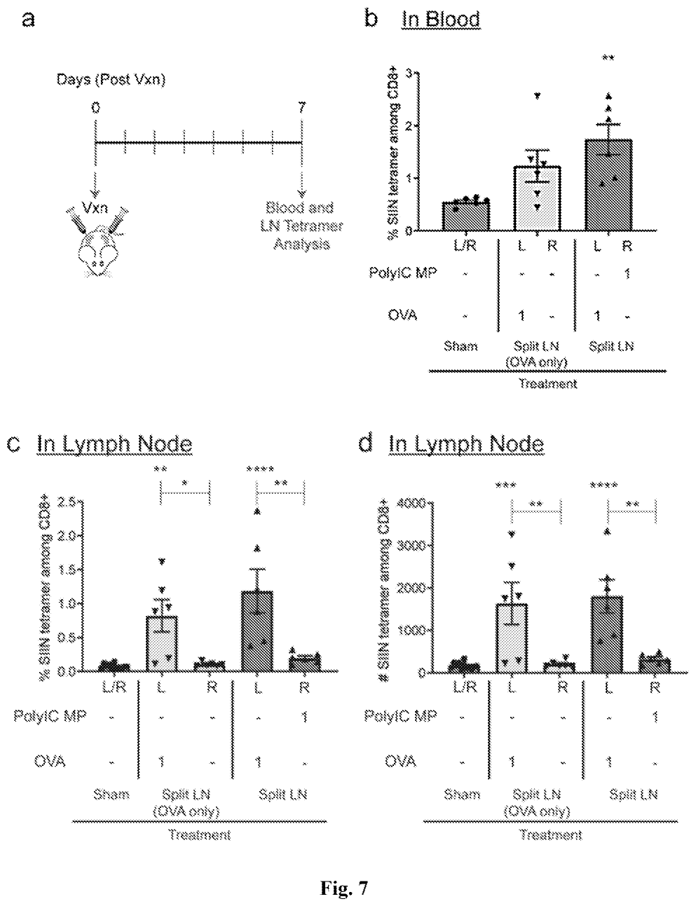

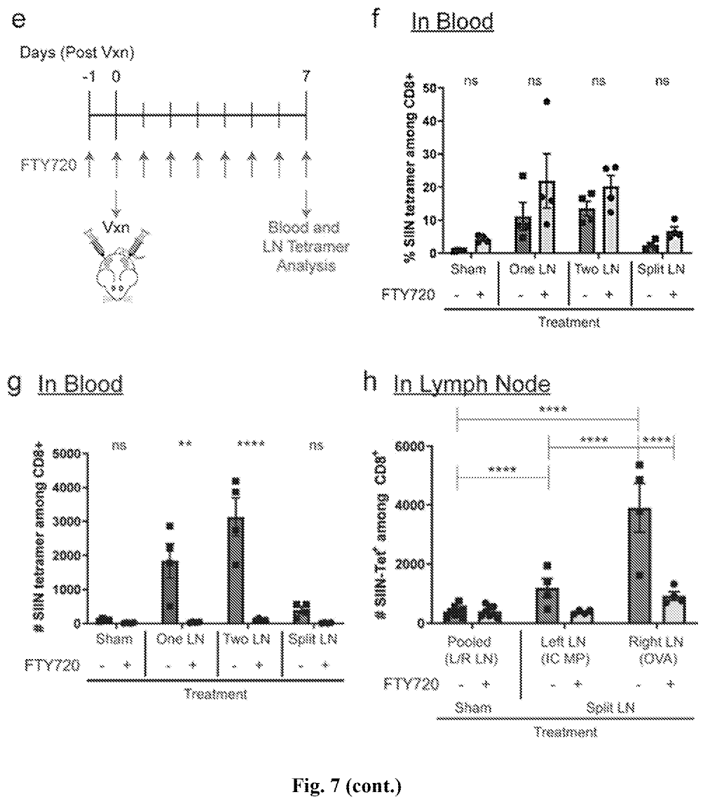

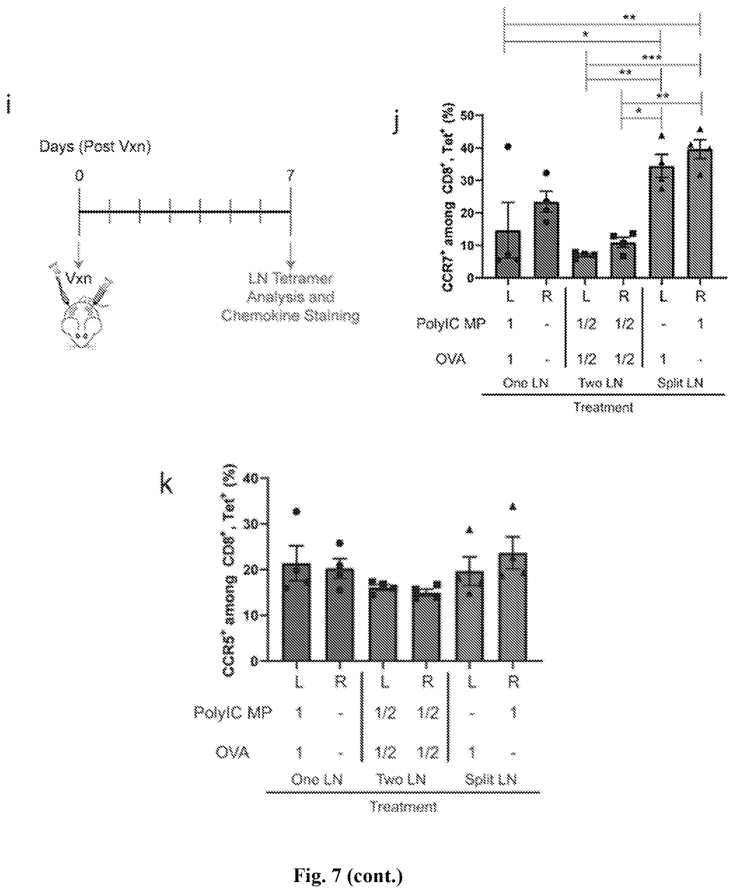

LN deposition of PolyIC MPs and cellular trafficking between LNs impact local and systemic antigen-specific responses. We next directly tested how the relative location of adjuvant depots controls the priming of functional T cells and the ability of these lymphocytes to leave LNs. In these studies, mice received OVA in the left inguinal LN, and either PBS or PolyIC MPs in the contralateral (right) inguinal LN (FIG. 7a). The regimen using OVA and PBS (“Split LN (OVA only)”) increased systemic antigen-specific T cells approximately 2-fold compared to Sham but did not reach the level of statistical significance (p=0.1494). However, treatment in the “Split LN” regimen with OVA and PolyIC MPs caused a statistically-significant increase in antigen-specific T cells in the blood by day 7; this represented an approximately 3-fold increase compared to the Sham baseline (FIG. 7b). Consistent with FIG. 2e, lf, these responses were still much lower compared to antigen and adjuvant MPs delivered to the same LN. Assessment of the local concentration of OVA-specific T cells in LNs (FIG. 7c,d) revealed increases in the frequency and number of these populations in any (left) LN that received antigen, but not locally in contralateral (right) LNs that received either PBS or PolyIC MPs (FIG. 7c,d). These data (FIG. 7b-d) are consistent with the results of the “Split LN” regimen in FIG. 6c,d discussed above, suggesting that T cells in LNs receiving only antigen can expand, but are not activated and thus unable to exit LNs. Importantly, this result also indicates adjuvant contained in depots administered to LNs is largely localized to those sites. This capability allows for focused dosing of strong immunomodulatory therapeutic components to locally generate active, tumor-specific populations while limiting systemic toxicity.

To directly investigate cellular trafficking, we administered daily systemic injections of a well-studied lymphocyte egress inhibitor, FTY720 (V. Brinkmann, J. G. Cyster, T. Hla, FTY720: sphingosine 1-phosphate receptor-1 in the control of lymphocyte egress and endothelial harrier function. Am J Transplant 4(7):1019-1025 (2004)), beginning one day prior to local LN injection (FIG. 7e). We first validated the system by confirming FTY720 did not preferentially skew antigen-specific T cell generation for each injection scheme (FIG. 7f). Following treatment with FTY720, the percentage of antigen-specific T cells in blood did not significantly change, validating the ability to generate antigen-specific T cells. In contrast, the number of antigen-specific T cells in blood was greatly diminished (FIG. 7g), confirming the effectiveness of the FTY720 treatment in blocking LN egress. Using the “Split LN” regimen without FTY720, as with the earlier experiments, the OVA-treated LN exhibited local increases in antigen-specific T cells within LNs (FIG. 7h, “Split LN—Right LN (OVA)”, dark green), but not in blood (FIG. 7g); this further indicates weak activation and an inability of these cells to migrate from the LN when adjuvant is restricted to other LNs using depots. In contrast, in mice receiving systemic FTY720, T cells in LNs receiving OVA (i.e., “Split—Right LN (OVA)”—light green) did not significantly expand in these tissues (FIG. 7h) or systemically in the blood (FIG. 7f,g). These results along with the data from FIG. 7c,d, further support that soluble PolyIC is not trafficking between nodes, regardless of whether FTY720 was administered. Therefore, one explanation for this finding is that FTY720 plays a role in limiting CD8⁺ expansion, while another that this difference may indicate in the absence of FTY720, instead of soluble PolyIC draining between LNs, other cellular trafficking occurs. In this second scenario, activated lymphocytes or APCs from a treated LN egress from these tissues to contralateral LNs containing OVA, resulting in T cell expansion in these LNs. This is analogous to the significant increases in systemic response observed when using PolyIC as a contralateral signal component compared with PBS (FIG. 7b). In all our studies, generation of OVA-specific T cells was only observed in LNs that received OVA, not contralateral nodes receiving PolyIC MPs. Thus, these data further indicate that free antigen did not drain significantly between nodes to promote antigen-specific T cell expansion in LNs treated only with PolyIC MPs.

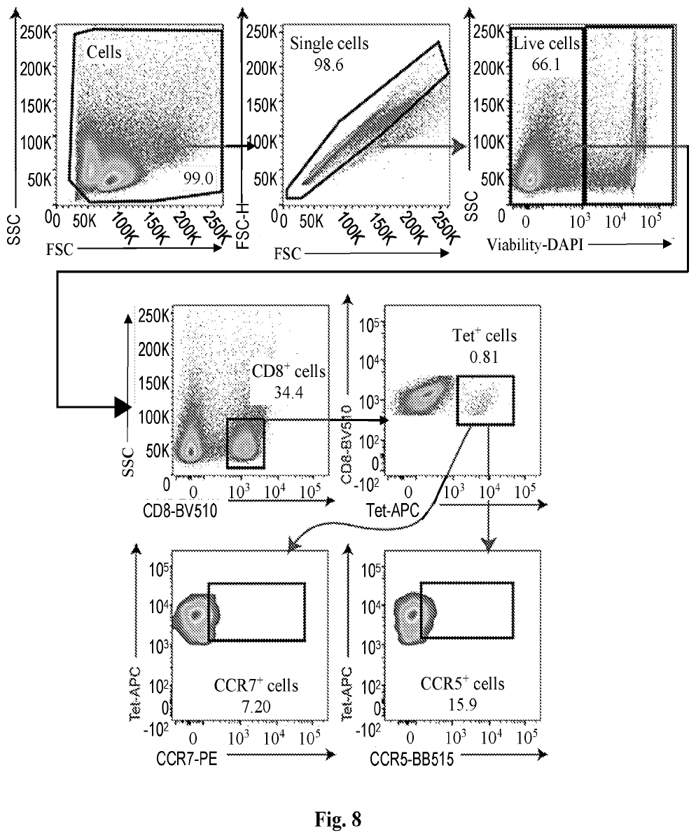

To further probe the effect of cellular trafficking on contralateral LN activation, mice were treated using the “One LN,” “Two LN” and “Split LN” treatment scheme as above and chemokine expression on antigen-specific T cells within the LN was evaluated 7 days after injection (FIG. 7i, FIG. 8). This chemokine staining was conducted to determine if immune cell homing signals, including C—C chemokine receptor type 5 and 7 (CCR5 and CCR7), are preferentially upregulated after injection. CCR7 is a receptor expressed on T cells to support homing to secondary lymphoid organs such as LNs, while CCR5 helps enable trafficking from LNs to peripheral tissues (R. Förster et al., CCR7 and its ligands: balancing immunity and tolerance. Nat Rev Immunol 8(5):362-371 (2008); M. Oppermann, Chemokine receptor CCR5: insights into structure, function, and regulation. Cell Signal 16(11):1201-1210 (2004)). In line with strong effector immune responses, the percentage of CCR7 expressed on antigen-specific CD8⁺ cells in LNs treated with both antigen and adjuvant decreased dramatically (FIG. 7j) when compared to other antigen-specific, CD8⁺ cells. “One LN” (Left LN) and “Two LN (Left and Right LN) both decreased CCR7 expression by 2 to 3-fold, respectively, compared to LNs receiving individual or no immune cues. This shift in CCR7 enables primed lymphocytes to leave LNs to seek out targets they have been activated against. Interestingly, treatment of a LN with a single immune cue (OVA or PolyIC MPs individually in the “Split LN”) or PBS treatment in the “One LN” treatment (right LN), showed no reduction in CCR7; these treatments each had similar CCR7 expression levels. Within these studies, there were minimal changes to CCR5 regardless of treatment (FIG. 7k).

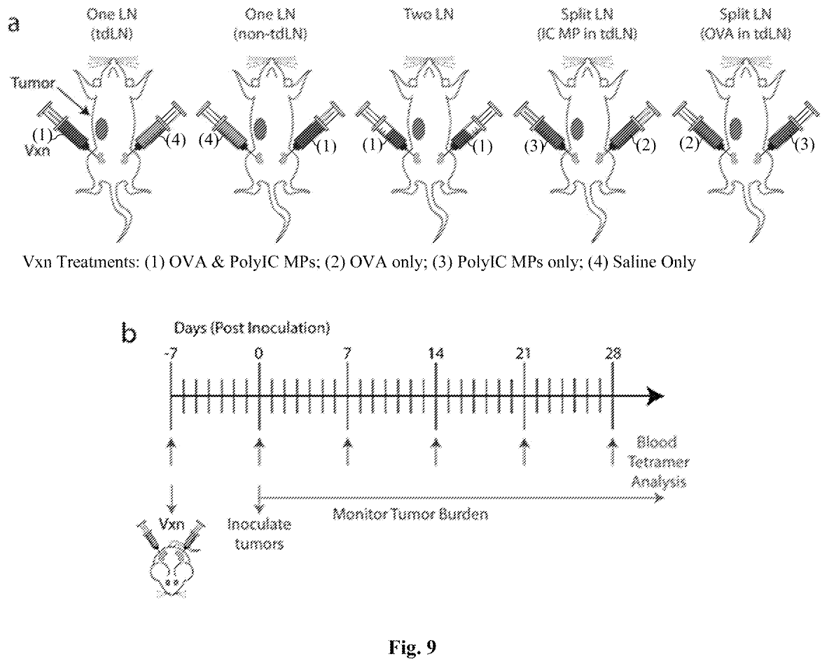

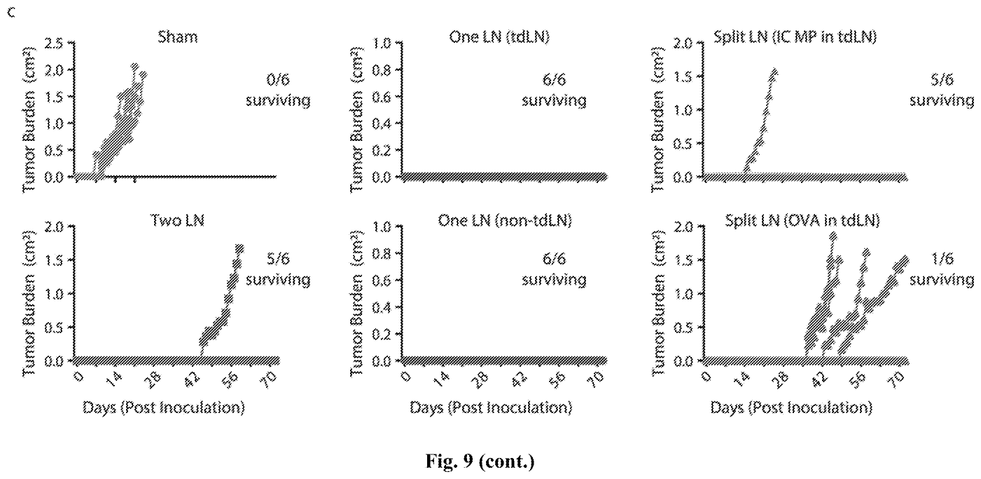

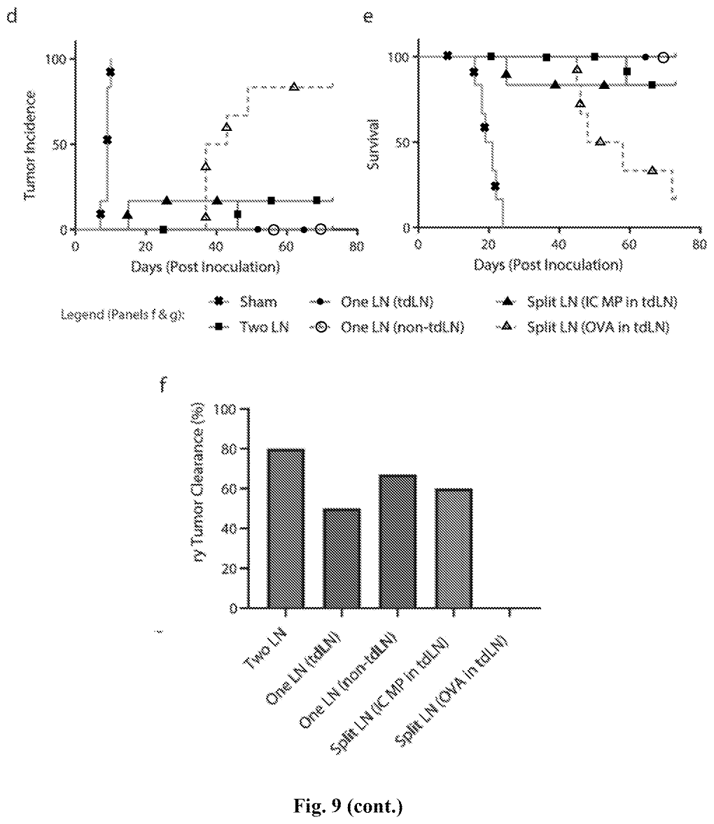

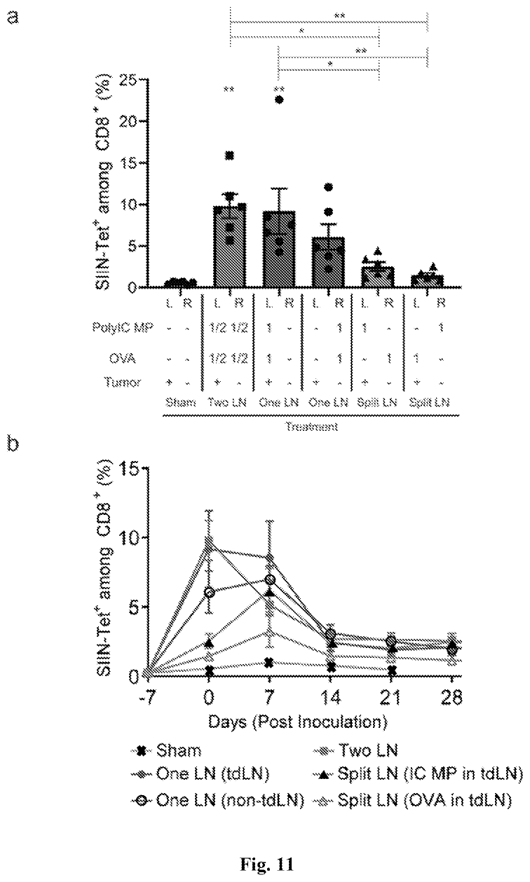

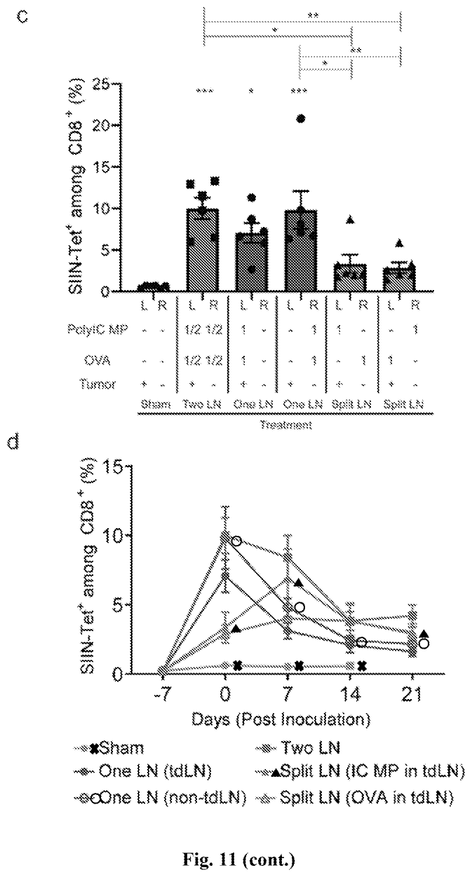

Distribution of antigen and adjuvant to LNs and tumors determines survival in murine melanoma models. Since the geographic distribution of signals resulted in dramatically different antigen-specific responses locally in LNs and systemically in blood, we next investigated these changes in a preventive cancer vaccination setting. Mice were vaccinated using the same “One LN,” “Two LN,” or “Split LN” treatment regimen as above and then inoculated subcutaneously with B16-OVA melanoma 7 days after treatment (FIG. 9a,b). In the case of the “One LN” and “Split LN” treatment regimens, we vaccinated mice in either tumor draining (tdLN) or non-tumor draining LN (non-tdLN) to test if vaccination proximity to the primary tumor influenced the resulting anti-tumor and antigen-specific response. In assessing tumor burden (FIG. 9c), the “Two LN” and either of the “One LN” regimens (tdLN or non-tdLN) generated immune responses that completely suppressed tumor growth. This efficacy resulted from a single injection and correlated to the significantly elevated percentage of antigen-specific T cells in the blood at the time of inoculation (FIG. 11a). Similar to trends seen in FIG. 2g, there was an increase in antigen-specific T cells in blood after 7 days which had a gradual contraction by day 28 (FIG. 11b).

When evaluating incidence (FIG. 9d) and survival (FIG. 9e), all treatment groups significantly differed from Sham treated LNs (Table 1). Intriguingly, the “Split LN” treatments resulted in divergent responses depending on which immune cue was delivered to the tdLN. When PolyIC MPs were delivered into the tdLN, tumor growth was suppressed (5/6 mice survived, FIG. 9c-e). However, when OVA was administered to the tdLN, most of the mice (5/6) developed tumors, albeit with a delayed onset of tumor formation. Thus, adjuvant depots localized to tdLNs created local environments able to generate potent antigen-specific tumor responses, while antigen sources (i.e., contralateral tumors or LNs) localized far from the LNs with adjuvant fails to provide protection. Along these lines, 14 days after vaccination the “Split LN” treatment with PolyIC MP in the tdLN exhibited increased antigen-specific cells in blood with greater than 5% Tee cells, while treatment with OVA in the tdLN exhibited less than 5% Tet⁺ cells (see FIG. 11b).

TABLE 1
B16-OVA multiple comparisons of survival curves.

| | Survival Difference | Incidence Difference |
|---|---|---|
| Treatment Group | from Sham | from Sham |
| Two LN | **, p < 0.01 | **, p < 0.01 |
| One LN (tdLN) | **, p < 0.01 | **, p < 0.01 |
| One LN (non-tdLN) | **, p < 0.01 | **, p < 0.01 |
| Split LN (IC MP in tdLN) | **, p < 0.01 | **, p < 0.01 |
| Split LN (OVA in tdLN) | **, p < 0.01 | **, p < 0.01 |

To investigate durability of tumor immunity, 13 weeks after the initial inoculation and well past the time of tumor growth and initial immune responses, surviving mice were rechallenged with 100,000 B16-OVA cells. No additional vaccinations were performed, a design requiring mice to have established durable tumor-specific responses solely from the prime therapeutic vaccine 98 days earlier. At least 50% of the mice survived and cleared a secondary tumor in each of the treated groups (“Two LN”, “One LN (tdLN)”, “One LN (non-tdLN)”, “Split LN (IC MP-tdLN)”), except for the “Split LN” with OVA in the tdLN (FIG. 9f, Table 2). In this group, the only mouse that did not succumb to the initial tumor did not survive a rechallenge. Together, the data from this rechallenge study indicates that adjuvant in tdLN ensures both tumor antigen and adjuvant are effectively presented. More broadly, these results indicate that potent, long-lasting anti-tumor responses can be achieved with a single treatment by creating appropriate relative signal geographies.

TABLE 2
Rechallenge of vaccinated mice surviving B16-OVA tumors.

| | # Mice | # Mice | % Mice |
|---|---|---|---|
| | Surviving | Surviving | Surviving |
| Treatment Group | 1° Challenge | 2° Challenge | 2° Challenge |
| Sham | 6 | 4 | 67% |
| Two LN | 5 | 4 | 80% |
| One LN (tdLN) | 6 | 3 | 50% |
| One LN (non-tdLN) | 6 | 4 | 67% |
| Split LN (IC MP in tdLN) | 5 | 3 | 60% |
| Split LN (OVA in tdLN) | 1 | 0 | 0% |

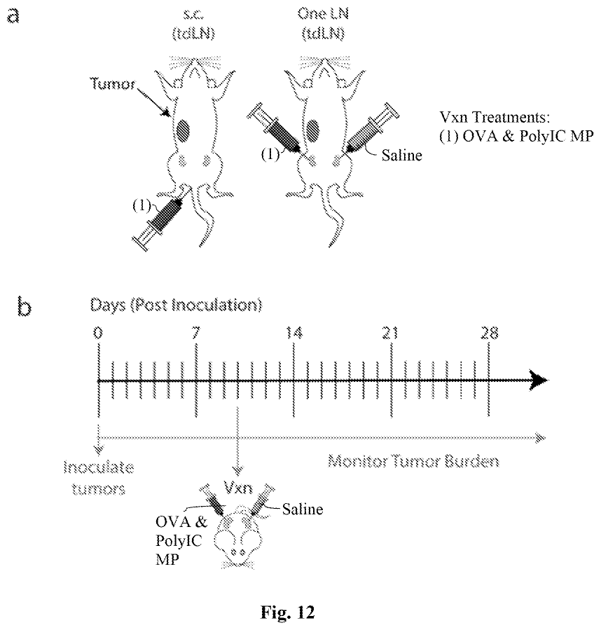

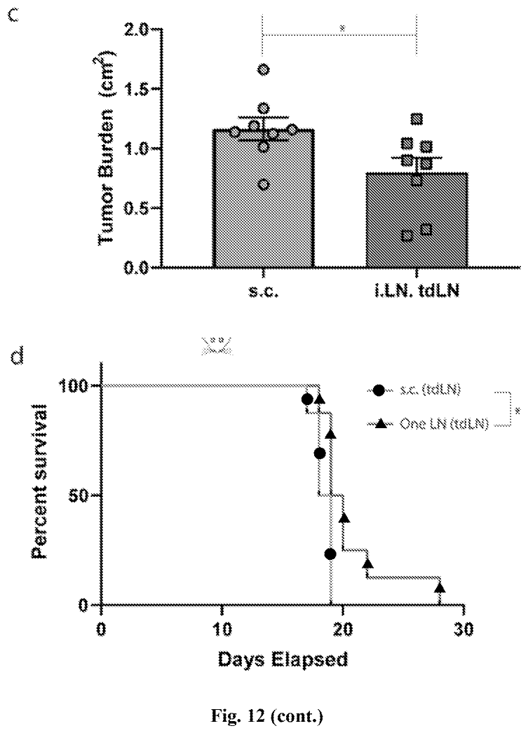

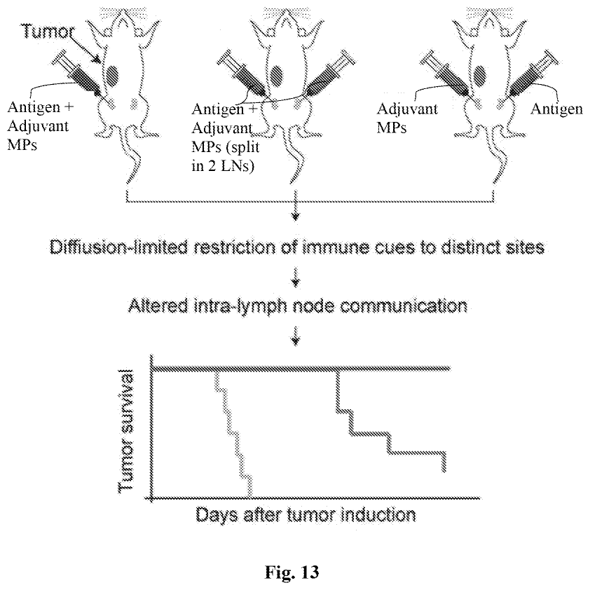

To further evaluate the capability of intra-LN injection for use as an immunotherapy delivery mechanism, an exploratory study was performed in a therapeutic melanoma model where mice were treated after the establishment of a tumor. In this more challenging setting, a high dose of B16F10 tumors (500,000/animal) were inoculated at Day 0. 10 days later—with clearly palpable tumors—mice were vaccinated via the “One LN” regimen in the tdLN, or using a benchmark of a matched dose given by subcutaneous injection (FIG. 12a,b). In these studies, at Day 17 when the first mouse succumbed to the tumor, there was a statistically-significant improvement in tumor burden for mice receiving the tdLN treatment versus the subcutaneous treatment (p-value=0.0340, FIG. 12c). Intra-LN vaccination on the tumor draining side also increased survival of mice compared to the subcutaneous benchmark (p-value=0.0172) (FIG. 12d). These data, while unoptimized in an aggressively growing tumor model, demonstrate intra-LN injection as an effective therapeutic modality, and create exciting opportunities for therapeutic regimens involving different location, dosing, or with combination therapies such as checkpoint inhibition.

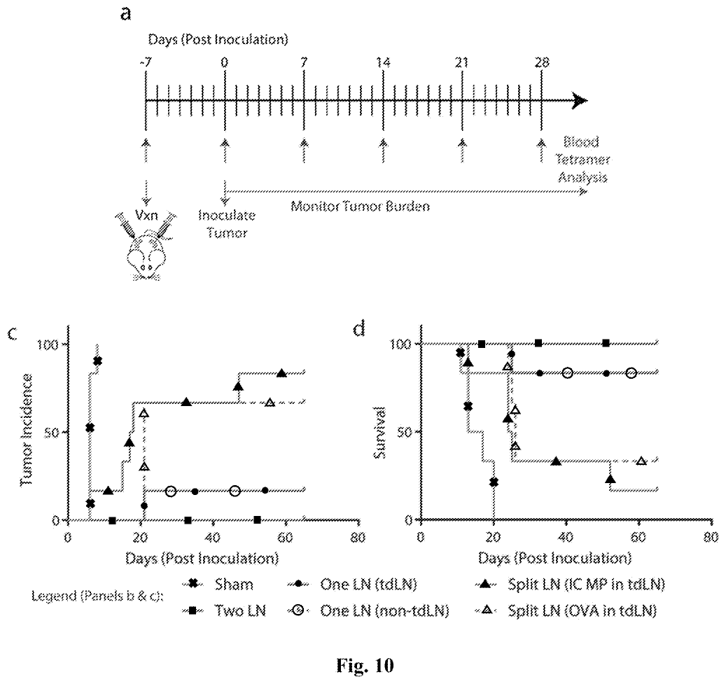

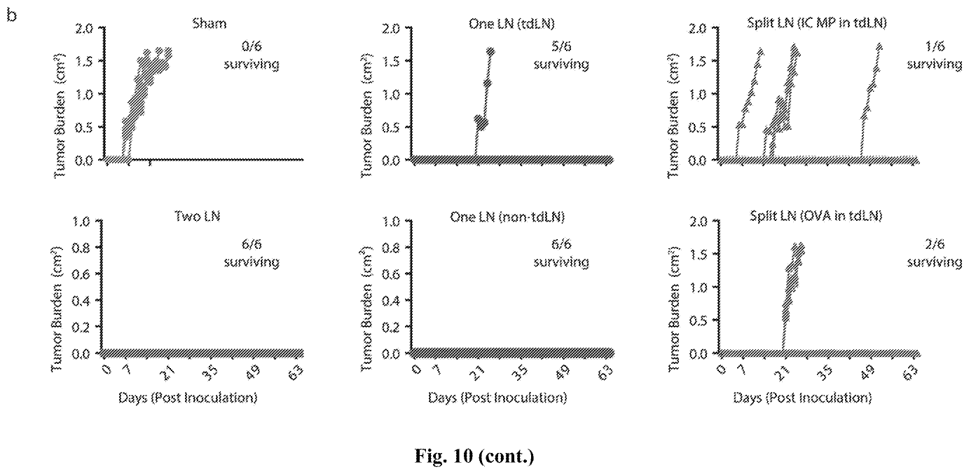

Co-delivery of antigen and adjuvant to LNs provides tumor protection and increased survival in a preventative lymphoma model. To determine if findings from the melanoma model were generalizable to another pre-clinical model, “One LN,” “Two LN,” and “Split LN” treatments were applied to a lymphoma model in which OVA is constitutively expressed. Mice were treated in a preventative manner as in FIG. 9a with treatments in tdLN and non-tdLN, EG7-OVA cancer cells were implanted 7 days after vaccination, and mice were evaluated daily for tumor growth and via weekly blood draws (up to day 28 post-inoculation) for antigen-specific T cell production (FIG. 10a). As with the findings from FIG. 2g and the previous melanoma experiments above, there were heightened antigen-specific T cell levels 7 days after treatment with all vaccine formulations (FIG. 11c). The “Two LN” or “One LN” treatment in either tdLN or non-tdLNs provided immune cues that generated anti-tumor immunity able to inhibit tumor growth and increase survival (17/18 mice surviving from these 3 groups) (FIG. 10b-d, Supplementary Table 3). However, in this lymphoma model, neither “Split LN” treatment (with PolyIC or OVA in the tdLN) was effective in protecting the mice from cancer with only 3/12 mice surviving in these treated groups (FIG. 10b-d). This indicates that compared to melanoma, even though the antigen-specific cells in circulation were heightened from “Split LN” treatment, these cells were not adequate to combat the lymphoma and provide protection, regardless of whether or not adjuvant was present in the tdLN.

TABLE 3
EG7-OVA multiple comparisons of survival curves.

| | Survival Difference | Incidence Difference |
|---|---|---|
| Treatment Group | from Sham | from Sham |
| Two LN | *, p < 0.05 | **, p < 0.01 |
| One LN (tdLN) | *, p < 0.05 | **, p < 0.01 |
| One LN (non-tdLN) | *, p < 0.05 | **, p < 0.01 |
| Split LN (IC MP in tdLN) | not significant (n.s.) | n.s. |
| Split LN (OVA in tdLN) | *, p < 0.05 | **, p < 0.01 |

The disclosed methods and systems provide tremendous opportunity in combining biomaterials with intra-LN injection methodologies (see, e.g., J. I. Andorko et al. (2015) Harnessing biomaterials to engineer the lymph node microenvironment for immunity or tolerance. AAPS J 17(2):323-328; Gammon, J. M. et al. (2015) Controlled delivery of a metabolic modulator promotes regulatory T cells and restrains autoimmunity. J Control Release 210:169-178; Tostanoski, L. H. et al. (2016) Reprogramming the Local Lymph Node Microenvironment Promotes Tolerance that Is Systemic and Antigen Specific. Cell Rep 16:2940-2952) for activating or enhancing immunological function in order to achieve disruptive therapeutic benefits. The use of larger, micron-size particles deposited in LNs creates diffusion limitations to spatially restrict signals in order to provide strong immune outcomes and thereby restrict toxic immunomodulatory signals to specific sites.

Intra-LN depots also provide unique opportunities to precisely study the role of therapeutic design parameters, such as relative geography of where signals are deposited and the local signal concentrations, and the impact of these variables on kinetics on immune function. Using fixed total doses of widely studied immune cues—antigen (OVA) and adjuvant (PolyIC), we achieved precision localization of the injected cargo to LNs. This allowed isolation of the role of individual and combined immune signals on local and systemic immunity. When investigating the local, intranodal response to treatment, as expected, uptake of PolyIC MPs by innate cells, in particular DCs and macrophages, led to activation of these cells. Interestingly, regardless of whether receiving the full dose of PolyIC MPs (e.g., “One LN” treatment or “Split LN” treatment) or half the number of particles per LN (e.g., in each LN for “Two LN” treatment), activation levels were comparable. This indicates a minimum threshold or particle concentration needed to drive tissue-wide activation of APCs. Using intra-LN delivery, titration studies may be performed to establish minimal concentrations suitable for local APC activation. Likewise, introduction of a lower dose (i.e., 0.5×) into more LNs (e.g., two), drove greater systemic, antigen-specific responses. This indicates another design rule that may be applied for immunotherapies: localizing cargo to several LNs at relatively low doses, instead of high doses at a single node. We also observed in the potent regimens of “Two LN” and “One LN” that despite strong systemic response, the local levels of antigen-specific T cells were relatively low. This may reflect migration of primed cells out of these tissues and into the periphery. Thus, the kinetics of signal and cell trafficking from treated to untreated LNs may be evaluated.

Introducing polymer depots or scaffolds directly into LNs influences stromal aspects of the local tissue environment, such as laminins involved in organizing microdomains important for immunity and tolerance (Warren, K. J. et al., Laminins affect T cell trafficking and allograft fate. J Clin Invest 124:2204-2218 (2014); Simon, T. & Bromberg, J. S. Regulation of the Immune System by Laminins. Trends in Immunology 38:858-871 (2017); Li, L. et al. The lymph node stromal laminin α5 shapes alloimmunity. J Clin Invest 130:2602-2619 (2020)). Physiochemical aspects may also be considered. For example, PLGA degradation products can alter local pH, which may influence local cell or signaling processes, although injection or carrier-induced effects were not observed. Taken together, combining biomaterial depots with local LN delivery creates potent immunotherapeutic treatments. Further, the disclosed techniques may be utilized for diagnosis of disease and/or the study of diseased tissue or cell migration as a function of signal concentration and location. Exploiting the unique capabilities of biomaterials to evaluate and/or affect immune function may be utilized for a wide variety of vaccination and immunotherapy applications aimed at LN targeting.

All identified publications and references mentioned in this specification are herein incorporated by reference to the same extent as if each individual publication was specifically and individually indicated to be incorporated by reference in its entirety. While the disclosure has been described in connection with exemplary embodiments thereof, it will be understood that it is capable of further modifications, and this application is intended to cover any variations, uses, or adaptations of the disclosure following, in general, the principles of the disclosure and including such departures from the present disclosure as come within known or customary practice within the art to which the disclosure pertains and as may be applied to the features hereinbefore set forth.

What is claimed is:

1. A method of eliciting an immune response to a cancer antigen in a subject, said method comprising introducing directly into at least one lymph node of the subject:
(a) a therapeutically effective amount of a composition comprising a cancer antigen, in combination with
(b) a carrier comprising an adjuvant such that an immune response to said cancer antigen is activated or enhanced in the subject.

2. The method of claim 1, wherein said carrier is a microparticle.

3. The method of claim 2, wherein said microparticle has a diameter of between about 1 μm and about 10 μm.

4. The method of any one of claims 2-3, wherein said microparticle is biodegradable and/or biocompatible.

5. The method of any one of claims 2-4, wherein said microparticle comprises a polymer material selected from the group consisting of poly(glycolide) (PGA), poly(L-lactide) (PLA), poly(beta-amino esters), and polyethylene glycol (PEG).

6. The method of any one of claims 1-5, wherein said cancer antigen and/or said adjuvant is introduced directly into the at least one lymph node of the subject via intra-lymph node injection.

7. The method of any one of claims 1-6, wherein said adjuvant is a toll-like receptor (TLR) agonist.

8. The method of claim 7, wherein said TLR agonist is polyinosinic:polycytidylic acid (Poly(I:C)).

9. The method of any one of claims 1-8, wherein said cancer antigen and said adjuvant are introduced into a single lymph node of the subject.

10. The method of any one of claims 1-8, wherein said cancer antigen and said adjuvant are introduced into at least two lymph nodes of the subject.

11. The method of any one of claims 1-8, wherein said cancer antigen is introduced into one or more lymph node(s) of the subject, and said adjuvant is introduced into one or more lymph node(s) of the subject different from the one or more lymph node(s) into which said cancer antigen is introduced.

12. The method of any one of claims 1-11, wherein said cancer antigen is associated with a cancer selected from the group consisting of breast cancer, prostate cancer, lung cancer, stomach cancer, colon cancer, rectal cancer, pancreatic cancer, liver cancer, ovarian cancer, throat cancer, esophageal cancer, bone cancer, melanoma, uterine cancer, testicular cancer, bladder cancer, kidney cancer, brain cancer, thyroid cancer, lymphoma, and leukemia.

13. The method of claim 12, wherein said cancer is a melanoma or a lymphoma.

14. The method of any one of claims 1-13, wherein the subject is a human.

15. The method of any one of claims 1-14, wherein the lymph node(s) into which said adjuvant is introduced is a/are tumor draining lymph node(s).

16. A method of treating or preventing cancer in a subject in need thereof, comprising introducing directly into at least one lymph node of the subject:
(a) a therapeutically effective amount of a composition comprising a cancer antigen, in combination with
(b) a carrier comprising an adjuvant such that an immune response to said cancer antigen is activated or enhanced in the subject.

17. The method of claim 16, wherein said carrier is a microparticle.

18. The method of claim 17, wherein said microparticle has a diameter of between about 1 μm and about 10 μm.

19. The method of any one of claims 17-18, wherein said microparticle is biodegradable and/or biocompatible.

20. The method of any one of claims 17-19, wherein said microparticle comprises a polymer material selected from the group consisting of poly(glycolide) (PGA), poly(L-lactide) (PLA), poly(beta-amino esters), and polyethylene glycol (PEG).

21. The method of any one of claims 16-20, wherein said cancer antigen and/or said adjuvant is introduced directly into the lymph node(s) of the subject via intra-lymph node injection.

22. The method of any one of claims 16-21, wherein said adjuvant is a toll-like receptor (TLR) agonist.

23. The method of claim 22, wherein said TLR agonist is polyinosinic:polycytidylic acid (Poly I:C).

24. The method of any one of claims 16-23, wherein said cancer antigen and said adjuvant are introduced into a single lymph node of the subject.

25. The method of any one of claims 16-23, wherein said cancer antigen and said adjuvant are introduced into at least two lymph nodes of the subject.

26. The method of any one of claims 16-23, wherein said cancer antigen is introduced into one or more lymph node(s) of the subject, and said adjuvant is introduced into one or more lymph node(s) of the subject different from the one or more lymph node(s) into which said cancer antigen is introduced.

27. The method of any one of claims 16-26, wherein said cancer antigen is associated with a cancer selected from the group consisting of breast cancer, prostate cancer, lung cancer, stomach cancer, colon cancer, rectal cancer, pancreatic cancer, liver cancer, ovarian cancer, throat cancer, esophageal cancer, bone cancer, melanoma, uterine cancer, testicular cancer, bladder cancer, kidney cancer, brain cancer, thyroid cancer, lymphoma, and leukemia.

28. The method of claim 27, wherein said cancer is melanoma or lymphoma.

29. The method of any one of claims 16-28, wherein the subject is a human.

30. The method of any one of claims 16-29, wherein the lymph node(s) into which said adjuvant is introduced is a/are tumor draining lymph node(s).

31. A pharmaceutical composition comprising a therapeutically effective amount of a cancer antigen, an adjuvant formulated to activate or enhance an immune response to the cancer antigen in a subject, and a pharmaceutically acceptable carrier, diluent, and/or excipient.

32. The pharmaceutical composition of claim 31, wherein said carrier is a microparticle.

33. The pharmaceutical composition of claim 32, wherein said microparticle has a diameter of between about 1 μm and about 10 μm.

34. The pharmaceutical composition of any one of claims 32-33, wherein said microparticle comprises a polymer material selected from the group consisting of poly(glycolide) (PGA), poly(L-lactide) (PLA), poly(beta-amino esters), and polyethylene glycol (PEG).

35. The pharmaceutical composition of any one of claims 31-34, which is formulated for administration via intra-lymph injection.

36. The pharmaceutical composition of any one of claims 31-35, wherein said adjuvant is a toll-like receptor (TLR) agonist.

37. The pharmaceutical composition of claim 36, wherein said TLR agonist is polyinosinic:polycytidylic acid (Poly PC).

38. The pharmaceutical composition of any one of claims 31-37, wherein said cancer antigen is associated with a cancer selected from the group consisting of breast cancer, prostate cancer, lung cancer, stomach cancer, colon cancer, rectal cancer, pancreatic cancer, liver cancer, ovarian cancer, throat cancer, esophageal cancer, bone cancer, melanoma, uterine cancer, testicular cancer, bladder cancer, kidney cancer, brain cancer, thyroid cancer, lymphoma, and leukemia.

39. The pharmaceutical composition of claim 38, wherein said cancer is melanoma or lymphoma.
